SEQUENCE LISTING (64 sequences)

SEQ ID NO: 1 - RNA, 63 nt, Mus musculus
  source 1..63: mol_type = genomic RNA
atcagtcaggctcctggctaaagttcagctttcagctgcatttagccaaagcctgactgtaag

SEQ ID NO: 2 - RNA, 20 nt, Mus musculus
  source 1..20: mol_type = genomic RNA
gggagtgcttcgtgcttctg

SEQ ID NO: 3 - RNA, 22 nt, Mus musculus
  source 1..22: mol_type = genomic RNA
catttagccaaagcctgactgt

SEQ ID NO: 4 - RNA, 64 nt, Mus musculus
  source 1..64: mol_type = genomic RNA
tcttgcatgtgggcctgtgtgctataatgtcacttgctaacagacgggctctcatgctgacaga

SEQ ID NO: 5 - RNA, 80 nt, Mus musculus
  source 1..80: mol_type = genomic RNA
ttcaatctgtggtactcaaactgtgtgacattttgttctttgtaagaagtgccgcagagtttgtagtgttgccgattgag

SEQ ID NO: 6 - RNA, 22 nt, Mus musculus
  source 1..22: mol_type = genomic RNA
agtgccgcagagtttgtagtgt

SEQ ID NO: 7 - RNA, 95 nt, Mus musculus
  source 1..95: mol_type = genomic RNA
ccctacacatccatctttgaggttacaacttccccagtagactgtttcctgacattccttgtgagttgttcctcacctggaagatgtggtggcgg

SEQ ID NO: 8 - RNA, 22 nt, Mus musculus
  source 1..22: mol_type = genomic RNA
tgagtattacatggccaatctc

SEQ ID NO: 9 - RNA, 22 nt, Mus musculus
  source 1..22: mol_type = genomic RNA
acctccatagtacctgcagcgt

SEQ ID NO: 10 - RNA, 59 nt, Mus musculus
  source 1..59: mol_type = genomic RNA
aaggactgggggattgggtgccttctttttaaaaaaagaaaaaacaccgtttcctccag

SEQ ID NO: 11 - RNA, 22 nt, Mus musculus
  source 1..22: mol_type = genomic RNA
tctgcatctaaggatatggtca

SEQ ID NO: 12 - RNA, 58 nt, Mus musculus
  source 1..58: mol_type = genomic RNA
tggaaggatgcctcttgcagatgtagcagaggaggtatctgcagaggcttccttggct

SEQ ID NO: 13 - RNA, 21 nt, Mus musculus
  source 1..21: mol_type = genomic RNA
tggaaggatgcctcttgcaga

SEQ ID NO: 14 - RNA, 21 nt, Mus musculus
  source 1..21: mol_type = genomic RNA
tagggccatctcatccagata

SEQ ID NO: 15 - RNA, 22 nt, Mus musculus
  source 1..22: mol_type = genomic RNA
cgtgttcacagcggaccttgat

SEQ ID NO: 16 - RNA, 20 nt, Mus musculus
  source 1..20: mol_type = genomic RNA
atcctgtccttgccctctct

SEQ ID NO: 17 - RNA, 23 nt, Mus musculus
  source 1..23: mol_type = genomic RNA
ggcttctttacagtgctgccttg

SEQ ID NO: 18 - RNA, 22 nt, Mus musculus
  source 1..22: mol_type = genomic RNA
cgagaattgcgtttggacaatc

SEQ ID NO: 19 - RNA, 20 nt, Mus musculus
  source 1..20: mol_type = genomic RNA
tgactgggaaggaaatggcc

SEQ ID NO: 20 - RNA, 21 nt, Mus musculus
  source 1..21: mol_type = genomic RNA
gggggcgtggaattatcgggt

SEQ ID NO: 21 - RNA, 21 nt, Mus musculus
  source 1..21: mol_type = genomic RNA
agtgtagcaggtaaaccagga

SEQ ID NO: 22 - RNA, 22 nt, Mus musculus
  source 1..22: mol_type = genomic RNA
cttacgaaagaatgaagagcaa

SEQ ID NO: 23 - RNA, 20 nt, Mus musculus
  source 1..20: mol_type = genomic RNA
tgtccttctctttcccccag

SEQ ID NO: 24 - RNA, 21 nt, Mus musculus
  source 1..21: mol_type = genomic RNA
gagcgaagcccaagcgccaga

SEQ ID NO: 25 - RNA, 58 nt, Mus musculus
  source 1..58: mol_type = genomic RNA
cttacgaaagaatgaagagcaagctgtgttctgggcttgtttcatttgttcgtgactc

SEQ ID NO: 26 - RNA, 65 nt, Mus musculus
  source 1..65: mol_type = genomic RNA
tgtgatctgggggctcagctgaggataagcccgccttgtcaccgctgtccttctctttcccccag

SEQ ID NO: 27 - RNA, 70 nt, Mus musculus
  source 1..70: mol_type = genomic RNA
gggagactgggcaggttgggttgaatgatttgatcatgtagtcacttaaataaccttatctgcccgatag

SEQ ID NO: 28 - RNA, 23 nt, Mus musculus
  source 1..23: mol_type = genomic RNA
actgggcaggttgggttgaatga

SEQ ID NO: 29 - RNA, 22 nt, Mus musculus
  source 1..22: mol_type = genomic RNA
aatgtgcctgtgcatctcttcc

SEQ ID NO: 30 - RNA, 61 nt, Mus musculus
  source 1..61: mol_type = genomic RNA
catggagtgtagcaggtaaaccaggaatctctctcaggtcttggttttcctgtttgcacag

SEQ ID NO: 31 - RNA, 74 nt, Mus musculus
  source 1..74: mol_type = genomic RNA
gctttttctgcatctaaggatatggtcatttaattgtttttttccatgatcatctccttaaatgcagaaaaagc

SEQ ID NO: 32 - RNA, 78 nt, Mus musculus
  source 1..78: mol_type = genomic RNA
tcacctgaatatgtgggggcattggtattttcaaagcagcattttgaatgtgcctgtgcatctcttcccacagtggtg

SEQ ID NO: 33 - RNA, 23 nt, Mus musculus
  source 1..23: mol_type = genomic RNA
agggacttttgggggcagatgtg

SEQ ID NO: 34 - RNA, 107 nt, Mus musculus
  source 1..107: mol_type = genomic RNA
tctcgcatggttcacgggaggacacacgcgtttctgttggtgattggggagcctgcctttgttcccacattgtaaggaactgcctgattttctctgaaatatggagt

SEQ ID NO: 35 - RNA, 22 nt, Mus musculus
  source 1..22: mol_type = genomic RNA
tggttcacgggaggacacacgc

SEQ ID NO: 36 - RNA, 22 nt, Mus musculus
  source 1..22: mol_type = genomic RNA
gcaaaggcgctgtttcccagtg

SEQ ID NO: 37 - RNA, 23 nt, Mus musculus
  source 1..23: mol_type = genomic RNA
atgggggagctgggattgggcgt

SEQ ID NO: 38 - RNA, 110 nt, Mus musculus
  source 1..110: mol_type = genomic RNA
acaccctcccccaggacagtaaagcacagtgatgaactctgagggcttttcacttcctctcggagttgctggttgctcactgggtctgtgaatgtgaatccatgtgggca

SEQ ID NO: 39 - RNA, 23 nt, Mus musculus
  source 1..23: mol_type = genomic RNA
ttctaggactttatagagcagag

SEQ ID NO: 40 - RNA, 126 nt, Mus musculus
  source 1..126: mol_type = genomic RNA
ggattctctgtgtagccttggctgtcctggcactcactctgtagaccaggctggccttgaactcaaaaattcacctgcctctgcctcccgagtgctgggactaaaggcatgcgctaccaatgcccg

SEQ ID NO: 41 - RNA, 22 nt, Mus musculus
  source 1..22: mol_type = genomic RNA
ctgactccctgcttctccgcag

SEQ ID NO: 42 - RNA, 22 nt, Mus musculus
  source 1..22: mol_type = genomic RNA
cttatgcaagattcccttctac

SEQ ID NO: 43 - RNA, 94 nt, Mus musculus
  source 1..94: mol_type = genomic RNA
tcgactctgggtttgaaccaaagctcgactcatggtttgaaccattacttaattcgtggtttgaaccatcactcgactcctggttcgaaccatc

SEQ ID NO: 44 - RNA, 23 nt, Mus musculus
  source 1..23: mol_type = genomic RNA
gggggccgatgcactgtaagaga

SEQ ID NO: 45 - RNA, 22 nt, Mus musculus
  source 1..22: mol_type = genomic RNA
tttgaaccatcactcgactcct

SEQ ID NO: 46 - RNA, 21 nt, Mus musculus
  source 1..21: mol_type = genomic RNA
aggttgcccatggtgtgttca

SEQ ID NO: 47 - RNA, 63 nt, Mus musculus
  source 1..63: mol_type = genomic RNA
tgccggagggatcgattgcttctccttttccagcaaaggatggaatctctccctctggtctga

SEQ ID NO: 48 - RNA, 22 nt, Mus musculus
  source 1..22: mol_type = genomic RNA
atggaatctctccctctggtct

SEQ ID NO: 49 - RNA, 22 nt, Mus musculus
  source 1..22: mol_type = genomic RNA
caggtggaacacgatgcaattt

SEQ ID NO: 50 - RNA, 93 nt, Mus musculus
  source 1..93: mol_type = genomic RNA
ttagaactgggaggtcttctaggactttatagagcagaggtttgcagcctgtgacactcagctcatggagacctaggtggccttgggttctga

SEQ ID NO: 51 - RNA, 21 nt, Mus musculus
  source 1..21: mol_type = genomic RNA
tccgggagggatggatgtgct

SEQ ID NO: 52 - RNA, 92 nt, Mus musculus
  source 1..92: mol_type = genomic RNA
gacgagcaataacagctagcgcgcatgcgtggaccagagacaaagctgtccccgtgtttacaaactgtgcgggtgaggatgtgtgctcctct

SEQ ID NO: 53 - RNA, 23 nt, Mus musculus
  source 1..23: mol_type = genomic RNA
gtaagtgagggcaagccttctgg

SEQ ID NO: 54 - RNA, 64 nt, Mus musculus
  source 1..64: mol_type = genomic RNA
ttggaatgggggagctgggattgggcgtggagcttggaggcctcaccctgtctgcgcccctcag

SEQ ID NO: 55 - RNA, 24 nt, Mus musculus
  source 1..24: mol_type = genomic RNA
agctgcgctgctcctggtaactgc

SEQ ID NO: 56 - RNA, 22 nt, Mus musculus
  source 1..22: mol_type = genomic RNA
gtgagtggccagggtggggctg

SEQ ID NO: 57 - RNA, 23 nt, Mus musculus
  source 1..23: mol_type = genomic RNA
tgactggcaccattctggataat

SEQ ID NO: 58 - RNA, 22 nt, Mus musculus
  source 1..22: mol_type = genomic RNA
tctggggaggagcgtagttaca

SEQ ID NO: 59 - RNA, 22 nt, Mus musculus
  source 1..22: mol_type = genomic RNA
ttgatgtccactgtgaccatag

SEQ ID NO: 60 - RNA, 57 nt, Mus musculus
  source 1..57: mol_type = genomic RNA
gtaagtgagggcaagccttctgggttcctaccatcccaggcggcctcagctctcact

SEQ ID NO: 61 - RNA, 77 nt, Mus musculus
  source 1..77: mol_type = genomic RNA
tgcagtgctctattcagattagtgccagtcatgtgaaatacatatgactggcaccattctggataatgtaatgctca

SEQ ID NO: 62 - RNA, 22 nt, Homo sapiens
  source 1..22: mol_type = genomic RNA
cgtgttcacagcggaccttgat

SEQ ID NO: 63 - RNA, 22 nt, Homo sapiens
  source 1..22: mol_type = genomic RNA
cttatgcaagattcccttctac

SEQ ID NO: 64 - RNA, 23 nt, Homo sapiens
  source 1..23: mol_type = genomic RNA
gggggccgatacactgtacgaga

(19) United States
(12) Patent Application Publication
(10) Pub. No.: US 2025/0263698 A1
(43) Pub. Date: Aug. 21, 2025

(54) EXOSOME THERAPY AND PROCESS TO IMPROVE FORMULATION AND PRODUCTION

(71) Applicants: STEADMAN PHILIPPON RESEARCH INSTITUTE, Vail, CO (US); COLORADO STATE UNIVERSITY RESEARCH FOUNDATION (CSURF), Fort Collins, CO (US)

(72) Inventors: Chelsea BAHNEY, Vail, CO (US); Nicole EHRHART, Ft. Collins, CO (US); Michael Terrance MULLEN, II, Vail, CO (US); Johnny HUARD, Vail, CO (US)

(21) Appl. No.: 18/692,770

(22) PCT Filed: Sep. 16, 2022
(86) PCT No.: PCT/US2022/076580

§ 371 Date: Mar. 15, 2024

Related U.S. Application Data
(60) Provisional application No. 63/245,729, filed on Sep. 17, 2021.

(51) Int. Cl. C12N 15/113; A61K 9/50

(52) U.S. Cl. CPC C12N 15/113; A61K 9/5068; C12N 2310/141

(57) ABSTRACT

The present disclosure provides compositions comprising exosomes comprising mechanoresponsive miRNAs and uses of the compositions. The disclosure also provides methods for preparing compositions comprising exosomes comprising mechanoresponsive miRNAs.

### CROSS-REFERENCE TO RELATED APPLICATIONS

This application is being filed on Sep. 16, 2022, as a PCT International Patent Application that claims priority to and the benefit of U.S. Provisional Patent Application No. 63/245,729, filed Sep. 17, 2021, the entire content of which is incorporated by reference herein.

### FIELD OF THE DISCLOSURE

The present disclosure is related to compositions comprising exosomes comprising mechanoresponsive miRNAs and uses of the compositions. The disclosure is also related to methods for preparing compositions comprising exosomes comprising mechanoresponsive miRNAs.

### REFERENCE TO A SEQUENCE LISTING XML

The instant application contains a Sequence Listing, which has been submitted electronically in XML format and is hereby incorporated by reference in its entirety. Said XML copy, created on Sep. 16, 2022, is named 18472_0022WOU1_SL.xml and is 58,366 bytes in size.

### BACKGROUND OF THE DISCLOSURE

Stem cell therapy transformed the field of regenerative medicine over the past several decades, as stem and progenitor cells have been utilized for a wide variety of therapeutic applications including the treatment of cardiovascular (Goradel, et al., 2021 J Cell Biochem 119 (1): 95-104), neurologic (Alessandrini, et al., 2019 South African Med J 109: 8b), and orthopedic (Grayson, et al., 2015 Nat Rev Endocrinol 11 (3): 140-150) diseases. While it was originally thought that stem cells impart their regenerative function by direct differentiation into new tissue, it is now widely accepted that the beneficial effects of stem and progenitor cells can largely be attributed to their paracrine activities (Gnecchi, et al., 2008 Circ Res 103 (11): 1204-1219; Baraniak and McDevitt, 2010 Regen Med 5 (1): 121-143; Huard, 2019 J Orthop Res 37 (6)). Mesenchymal stem cells (MSCs) are capable of releasing bioactive molecules, including immunomodulatory and trophic factors, to establish a microenvironment that promotes tissue repair and regeneration by resident cells (Caplan and Correa, 2011 Cell Stem Cell 9 (1); Caplan, 2009 Tissue Eng—Pt B Rev 15 (2): 195-200). In support of this concept, less than 1% of administered MSCs remain viable up to one week following intravenous injection (Eggenhofer, et al., 2012 Front Immunol 3 (SEP; Lee, et al., 2009 Cell Stem Cell 5 (1): 54-63) and do not readily differentiate in vivo (Caplan, 2017 Stem Cells Transl Med 6 (6)), suggesting that the paracrine functions of MSCs are responsible for the benefits of MSC administration (Parekkadan and Milwid, 2010 Ann Rev Biomed Eng 12:87-117).

Extracellular vesicles produced by MSCs have also been recognized as a major source of these pro-regenerative paracrine functions (Rani, et al., 2015 Mol Ther 23 (5): 812-823; Wiklander, et al., 2019 Sci Transl Med 11 (492): 8521), Exosomes, a subtype of extracellular vesicles that have garnered significant interest in recent years, originate within intracellular multivesicular bodies and are released into the extracellular space as nanosized (30-150 nm) membrane-bound vesicles upon fusion with the cell membrane. These exosomes can either be endocytosed by nearby cells or can enter circulation to be transported throughout the body. Exosomes contain diverse cargo including nucleic acids, proteins, and lipids, many of which are biologically active and can alter the function of recipient cells. This exosomal cargo is dependent on the cell type of origin, as well as the local microenvironment and physiological state of the originating cell (Phinney and Pittenger, 2017 Stem Cells 35 (4): 851-858). A notable component of exosomes includes micro-RNAs (miRNAs), which are small, 20-25-nucleotide RNAs that act as negative regulators of gene expression by binding the 3′UTR of mRNAs to inhibit translation and modulate cell function (Zhang, et al., 2015 Proteomics Bioinforma 13 (1): 17-24; Kalluri and LeBleu, 2020 Science 80 (367): 6478).

The ability of exosomes to deliver bioactive molecules to cells has been widely recognized for its therapeutic potential. Within the field of regenerative medicine, exosomes hold several advantages over traditional cell-based therapies. Exosomes can be isolated from desirable cell types, such as MSCs, or can be engineered to contain pro-regenerative miRNAs and other beneficial cargo to promote specified biological functions upon administration to a patient. In contrast to stem cell therapy, exosomes are currently understood to have minimal to no risk of immune rejection (Yu, et al., 2014 Int J Mol Sci 15 (3): 4142-4157). Exosomes are also non-dividing and cannot directly form tumors, whereas stem cells may have a tumorigenic potential (Lazennec and Jorgensen, 2008 Stem Cells 26 (6): 1387-1394). Additionally, the use of MSCs as a therapeutic presents several manufacturing and regulatory challenges that exosomes may circumvent (Mendt, et al., 2019 Bone Marrow Transplant 54 (2): 789-792), thus presenting an alternative therapeutic that replicates the benefits of stem cell therapy while minimizing the associated risks and manufacturing challenges.

One such area where exosomes hold therapeutic promise is musculoskeletal regeneration (Williams and Ehrhart, 2022 J Am Vet Med Assoc 260 (7): 683-689). During aging, the tissue quality and regenerative capacity of skeletal muscle and bone are severely compromised (Carosio, et al., 2011 Aging Res Rev 10 (1); Domingues-Faria, et al., 2016 Aging Res Rev 26). This contributes to age-related muscle loss and dysfunction, termed sarcopenia, as well as osteoporosis, which together are linked to frailty, increased risk of fracture, and increased mortality (Fielding, et al., 2011 J Am Med Dir Assoc 12 (4): 249-256). Cellular crosstalk between muscle and bone is known to occur, as sarcopenia and osteoporosis frequently occur together (Reginster, et al., 2016 Curr Opin Clin Nutr Metab Care 19 (1); Edwards, et al., 2015 Bone 80:126-130). Moreover, it has been demonstrated that paracrine factors released from skeletal muscle and muscle stem cells contribute to bone fracture repair (Julien, et al., 2021 Nat Commun 12 (1); Abou-Khalil, et al., 2015 Stem Cells 33 (5): 1501-1511).

Within the next 30 years, the global population of individuals aged 65 and over is projected to double, reaching 1.5 billion by the year 2050. To meet the medical challenges faced by an aging population, it is critical to develop therapeutics that promote the maintenance, repair, and regeneration of muscle and bone. While resistance exercise is an effective strategy to maintain bone density (Benedetti, et al., 2018 Biomed Res Int https://pubmed.ncbi.nlm.nih.gov/30671455/) and skeletal muscle function during the aging process (Vincent, et al., 2002 J Am Geriatr Soc 50 (6): 1100-1107; Law, et al., 2016 Ann Rev Gerontol Geriatr 36 (1)), there are currently no available pharmacological options to mitigate the age-related decline in muscle regenerative capacity. Mechanical strain of skeletal muscle has been hypothesized to mediate the benefits of resistance exercise on the functional capacity of muscle (Ambrosio, et al., 2009 Am J Phys Med Rehab 88 (2)). Additionally, several studies have shown that in vivo mechanical strain improves muscle regeneration and functional recovery following injury (Cezar, et al., 2016 PNAS USA 113 (6): 1534-1539; Ambrosio, et al., 2010 Tissue Eng—Pt A 16 (3): 839-849; Kohno, et al., 2012 J Appl Physiol 112 (10): 1773-1782). Yet, the mechanisms underlying how mechanical strain improves skeletal muscle regeneration at a molecular level remain unclear. Exercise has recently been found to produce a significant increase in circulating exosomes (Fruehbeis, et al., 2015 J Extracell Vesicles 4 (1); Whitham, et al., 2018 Cell Metab 27 (1): 237-251), suggesting that exosomes may mediate the benefits of exercise on both muscle and bone health.

miRNA, which is a common non-coding RNA, can target various mRNAs to regulate their physiological activities. Therefore, miRNAs play an important role in various physiological and pathological processes, and so they have been proposed as a powerful tool to treat different diseases efficiently. However, the characteristic of miRNA degradation in vivo limits its further clinical application. Exosomes have the advantage of crossing the biological barrier and achieving long-distance communication with cells, so they are excellent vectors for miRNAs. By studying the biogenesis of exosomes, the way for loading miRNAs, the mechanism of targeting, and disease occurrence and development, it is confirmed that exosomes can enrich specific endogenous miRNAs and regulate a variety of physiological activities, such as promoting cancer cell apoptosis, regulating lipid metabolism and promoting angiogenesis. It is shown that exosomes loaded with miRNAs have good performance in the fields of cancer, neurodegenerative diseases, cardiovascular disease treatment, and regenerative medicine. (Fang, et al., 2022 Chinese Chem Lett 4:1693-1704)

### BRIEF SUMMARY OF THE DISCLOSURE

it has previously been demonstrated that in vitro mechanical strain (i.e., ex vivo exercise) and in vivo treadmill exercise can further increase the regenerative capacity of muscle progenitor cells when transplanted into injured skeletal muscle, but the mechanism underlying this observation is unknown. Described herein is the determination of whether exosomes produced by mechanically strained muscle progenitor cells contribute to this increased regenerative capacity. Mechanical strain may alter the secretome of myoblasts to favor the secretion of exosomes with regenerative functions. Ultimately, the instant disclosure guides strategies for the development of exosome therapeutics targeting the age-associated decline in musculoskeletal repair and regeneration.

Exercise triggers the release of exosomes carrying such metabolic products as, without limitation, microRNA (miRNA), messenger RNA (mRNA), cytokines such as chemokines, interleukins and lymphokines, and other proteins such as growth factors and the like. The thus released exosomes carry a specific miRNA signature. That signature may induce gene expression changes in the subject. The molecular characterization of exercise-induced exosomal miRNAs and their effects may lend to the identification of new therapeutic strategies to alleviate aging-related ailments.

In one aspect, the disclosure provides a composition comprising exosomes comprising at least one mechanoresponsive miRNA. In one embodiment, the exosomes are derived from mechanosensitive cells. In a further embodiment, the mechanosensitive cells are selected from the group consisting of muscle cells, stem cells (e.g., mesenchymal stem cells, muscle-derived stem cells), muscle fiber (e.g., skeletal muscle fiber), endothelial cells (e.g., blood vessel cells), nerve cells, bone cells (e.g., osteoblasts, osteoclasts, osteocytes), chondrocytes, and cardiomyocytes. In still further embodiments, the cells are from a subject of any age (e.g., embryonic to infant to child to adolescent to young to middle age to old). Thus, for example, the muscle-derived stem cells may be from an elderly subject.

In one embodiment of a composition according to the disclosure, the exosomes are isolated from a biological sample of a mammalian subject after exercise. In another embodiment, the sample is muscle (e.g., skeletal muscle)—by biopsy, bone marrow, or blood.

In one embodiment of a composition according to the disclosure, the exosomes are isolated from a cell sample (i.e., in vitro) after ex vivo exercise. In another embodiment, the ex vivo exercise is achieved by a bioreactor creating mechanical strain on cells in the sample.

In additional embodiments, the exercise is low intensity or high intensity.

In one embodiment of a composition according to the disclosure, the exosomes are artificially synthesized.

In another embodiment, a composition according to the disclosure is substantially free of cells.

In one embodiment of a composition according to the disclosure, the at least one mechanoresponsive miRNA is pro-regenerative. Pro-regenerative, as used herein, refers to promoting proliferation and differentiation. In one embodiment, pro-regenerative refers to promoting proliferation and differentiation of muscle. In another embodiment, pro-regenerative refers to promoting proliferation and differentiation of bone. In another embodiment, some of the miRNAs the exosome comprises are pro-regenerative. In still another embodiment, all of the miRNAs the exosome comprises are pro-regenerative.

In one embodiment of a composition according to the disclosure, the at least one mechanoresponsive miRNA is the human equivalent of an miRNA selected from the group consisting of: mmu-mir-344b (SEQ ID NO: 1), mmu-miR-3060-5p (SEQ ID NO: 2), mmu-miR-344b-3p (SEQ ID NO: 3), mmu-mir-12184 (SEQ ID NO: 4), mmu-mir-293 (SEQ ID NO: 5), mmu-miR-293-3p (SEQ ID NO: 6), mmu-mir-804 (SEQ ID NO: 7), mmu-miR-496a-3p (SEQ ID NO: 8), mmu-miR-1930-5p (SEQ ID NO: 9), mmu-mir-7119 (SEQ ID NO: 10), mmu-miR-1950 (SEQ ID NO: 11), mmu-mir-12201 (SEQ ID NO: 12), mmu-miR-12201-5p (SEQ ID NO: 13), mmu-miR-3109-3p (SEQ ID NO: 14), mmu-miR-124-5p (SEQ ID NO: 15), mmu-miR-7681-5p (SEQ ID NO: 16), mmu-miR-103-1-5p (SEQ ID NO: 17), mmu-miR-219c-3p (SEQ ID NO: 18), mmu-miR-6961-5p (SEQ ID NO: 19), mmu-miR-6244 (SEQ ID NO: 20), mmu-miR-7065-5p (SEQ ID NO: 21), mmu-miR-7675-5p (SEQ ID NO: 22), mmu-miR-7117-3p (SEQ ID NO: 23), mmu-miR-6546-3p (SEQ ID NO: 24), mmu-mir-7675 (SEQ ID NO: 25), mmu-mir-7117 (SEQ ID NO: 26), mmu-mir-6905 (SEQ ID NO: 27), mmu-miR-6905-5p (SEQ ID NO: 28), mmu-miR-5129-3p (SEQ ID NO: 29), mmu-mir-7065 (SEQ ID NO: 30), mmu-mir-1950 (SEQ ID NO: 31), mmu-mir-5129 (SEQ ID NO: 32), mmu-miR-365-1-5p (SEQ ID NO: 33), mmu-mir-6378 (SEQ ID NO: 34), mmu-miR-6378 (SEQ ID NO: 35), mmu-miR-6403 (SEQ ID NO: 36), mmu-miR-7023-5p (SEQ ID NO: 37), mmu-mir-6539 (SEQ ID NO: 38), mmu-miR-1929-5p (SEQ ID NO: 39), mmu-mir-3470b (SEQ ID NO: 40), mmu-miR-3077-3p (SEQ ID NO: 41), mmu-miR-491-3p (SEQ ID NO: 42), mmu-mir-434 (SEQ ID NO: 43), mmu-miR-128-2-5p (SEQ ID NO: 44), mmu-miR-434-3p (SEQ ID NO: 45), mmu-miR-496a-5p (SEQ ID NO: 46), mmu-mir-12203 (SEQ ID NO: 47), mmu-miR-12203-3p (SEQ ID NO: 48), mmu-miR-363-5p (SEQ ID NO: 49), mmu-mir-1929 (SEQ ID NO: 50), mmu-miR-7034-5p (SEQ ID NO: 51), mmu-mir-6409 (SEQ ID NO: 52), mmu-miR-5617-5p (SEQ ID NO: 53), mmu-mir-7023 (SEQ ID NO: 54), mmu-miR-2139 (SEQ ID NO: 55), mmu-miR-3102-5p (SEQ ID NO: 56), mmu-miR-871-3p (SEQ ID NO: 57), mmu-miR-8108 (SEQ ID NO: 58), mmu-miR-3073a-3p (SEQ ID NO: 59), mmu-mir-5617 (SEQ ID NO: 60), and mmu-mir-871 (SEQ ID NO: 61).

In another embodiment, the at least one mechanoresponsive miRNA is the human equivalent of an miRNA selected from the group consisting of: mmu-mir-344b (SEQ ID NO: 1), mmu-miR-3060-5p (SEQ ID NO: 2), mmu-miR-344b-3p (SEQ ID NO: 3), mmu-mir-12184 (SEQ ID NO: 4), mmu-mir-293 (SEQ ID NO: 5), mmu-miR-293-3p (SEQ ID NO: 6), mmu-mir-804 (SEQ ID NO: 7), mmu-miR-496a-3p (SEQ ID NO: 8), mmu-miR-1930-5p (SEQ ID NO: 9), mmu-mir-7119 (SEQ ID NO: 10), mmu-miR-1950 (SEQ ID NO: 11), mmu-mir-12201 (SEQ ID NO: 12), mmu-miR-12201-5p (SEQ ID NO: 13), mmu-miR-3109-3p (SEQ ID NO: 14), mmu-miR-124-5p (SEQ ID NO: 15), mmu-miR-7681-5p (SEQ ID NO: 16), mmu-miR-103-1-5p (SEQ ID NO: 17), mmu-miR-219c-3p (SEQ ID NO: 18), mmu-miR-6961-5p (SEQ ID NO: 19), mmu-miR-6244 (SEQ ID NO: 20), mmu-miR-7065-5p (SEQ ID NO: 21), mmu-miR-7675-5p (SEQ ID NO: 22), mmu-miR-7117-3p (SEQ ID NO: 23), mmu-miR-6546-3p (SEQ ID NO: 24), mmu-mir-7675 (SEQ ID NO: 25), mmu-mir-7117 (SEQ ID NO: 26), mmu-mir-6905 (SEQ ID NO: 27), mmu-miR-6905-5p (SEQ ID NO: 28), mmu-miR-5129-3p (SEQ ID NO: 29), mmu-mir-7065 (SEQ ID NO: 30), mmu-mir-1950 (SEQ ID NO: 31), mmu-mir-5129 (SEQ ID NO: 32), and mmu-miR-365-1-5p (SEQ ID NO: 33).

In still another embodiment, the at least one mechanoresponsive miRNA is the human equivalent of an miRNA selected from the group consisting of: mmu-mir-6378 (SEQ ID NO: 34), mmu-miR-6378 (SEQ ID NO: 35), mmu-miR-6403 (SEQ ID NO: 36), mmu-miR-7023-5p (SEQ ID NO: 37), mmu-mir-6539 (SEQ ID NO: 38), mmu-miR-1929-5p (SEQ ID NO: 39), mmu-mir-3470b (SEQ ID NO: 40), mmu-miR-3077-3p (SEQ ID NO: 41), mmu-miR-491-3p (SEQ ID NO: 42), mmu-mir-434 (SEQ ID NO: 43), mmu-miR-128-2-5p (SEQ ID NO: 44), mmu-miR-434-3p (SEQ ID NO: 45), mmu-miR-496a-5p (SEQ ID NO: 46), mmu-mir-12203 (SEQ ID NO: 47), mmu-miR-12203-3p (SEQ ID NO: 48), mmu-miR-363-5p (SEQ ID NO: 49), mmu-mir-1929 (SEQ ID NO: 50), mmu-miR-7034-5p (SEQ ID NO: 51), mmu-mir-6409 (SEQ ID NO: 52), mmu-miR-5617-5p (SEQ ID NO: 53), mmu-mir-7023 (SEQ ID NO: 54), mmu-miR-2139 (SEQ ID NO: 55), mmu-miR-3102-5p (SEQ ID NO: 56), mmu-miR-871-3p (SEQ ID NO: 57), mmu-miR-8108 (SEQ ID NO: 58), mmu-miR-3073a-3p (SEQ ID NO: 59), mmu-mir-5617 (SEQ ID NO: 60), and mmu-mir-871 (SEQ ID NO: 61).

In further embodiments of a composition according to the disclosure, “at least one” in the context of the miRNAs may mean one, more than one, or all of each of the above lists of miRNAs.

In one embodiment, a composition according to the disclosure is for use in promoting regeneration in a subject.

In one embodiment, a composition according to the disclosure is for use in the treatment of a musculoskeletal condition or disorder.

In one aspect, the disclosure provides a method of preparing a composition comprising exosomes comprising at least one mechanoresponsive miRNA, the method comprising subjecting a mammal to exercise, obtaining a biological sample from the mammal, and isolating the exosomes from the sample. In one embodiment, the sample is muscle (e.g., skeletal muscle)—by biopsy, bone marrow, or blood.

In another aspect, the disclosure provides a method of preparing a composition comprising exosomes comprising at least one mechanoresponsive miRNA, the method comprising exercising cells (e.g., mechanosensitive cells) ex vivo and isolating the exosomes from the cells. In one embodiment, the ex vivo exercise is achieved by a bioreactor creating mechanical strain on cells in the sample. Exercise, in these embodiments, is mechanical loading that simulates endogenous loading. For muscle cells and the like, mechanical loading by, for example, comprise axial loading and/or uniaxial loading. For chondrocytes and the like, for example, mechanical loading may comprise compressive loading. Mechanical loading may also, in certain embodiments include shear loading.

In additional embodiments, the exercising is at low intensity or at high intensity.

In one embodiment of a method according to the disclosure, the at least one mechanoresponsive miRNA is pro-regenerative. In another embodiment, some of the miRNAs the exosome comprises are pro-regenerative. In still another embodiment, all of the miRNAs the exosome comprises are pro-regenerative.

In one embodiment of a method according to the disclosure, the at least one mechanoresponsive miRNA is the human equivalent of an miRNA selected from the group consisting of: mmu-mir-344b (SEQ ID NO: 1), mmu-miR-3060-5p (SEQ ID NO: 2), mmu-miR-344b-3p (SEQ ID NO: 3), mmu-mir-12184 (SEQ ID NO: 4), mmu-mir-293 (SEQ ID NO: 5), mmu-miR-293-3p (SEQ ID NO: 6), mmu-mir-804 (SEQ ID NO: 7), mmu-miR-496a-3p (SEQ ID NO: 8), mmu-miR-1930-5p (SEQ ID NO: 9), mmu-mir-7119 (SEQ ID NO: 10), mmu-miR-1950 (SEQ ID NO: 11), mmu-mir-12201 (SEQ ID NO: 12), mmu-miR-12201-5p (SEQ ID NO: 13), mmu-miR-3109-3p (SEQ ID NO: 14), mmu-miR-124-5p (SEQ ID NO: 15), mmu-miR-7681-5p (SEQ ID NO: 16), mmu-miR-103-1-5p (SEQ ID NO: 17), mmu-miR-219c-3p (SEQ ID NO: 18), mmu-miR-6961-5p (SEQ ID NO: 19), mmu-miR-6244 (SEQ ID NO: 20), mmu-miR-7065-5p (SEQ ID NO: 21), mmu-miR-7675-5p (SEQ ID NO: 22), mmu-miR-7117-3p (SEQ ID NO: 23), mmu-miR-6546-3p (SEQ ID NO: 24), mmu-mir-7675 (SEQ ID NO: 25), mmu-mir-7117 (SEQ ID NO: 26), mmu-mir-6905 (SEQ ID NO: 27), mmu-miR-6905-5p (SEQ ID NO: 28), mmu-miR-5129-3p (SEQ ID NO: 29), mmu-mir-7065 (SEQ ID NO: 30), mmu-mir-1950 (SEQ ID NO: 31), mmu-mir-5129 (SEQ ID NO: 32), mmu-miR-365-1-5p (SEQ ID NO: 33), mmu-mir-6378 (SEQ ID NO: 34), mmu-miR-6378 (SEQ ID NO: 35), mmu-miR-6403 (SEQ ID NO: 36), mmu-miR-7023-5p (SEQ ID NO: 37), mmu-mir-6539 (SEQ ID NO: 38), mmu-miR-1929-5p (SEQ ID NO: 39), mmu-mir-3470b (SEQ ID NO: 40), mmu-miR-3077-3p (SEQ ID NO: 41), mmu-miR-491-3p (SEQ ID NO: 42), mmu-mir-434 (SEQ ID NO: 43), mmu-miR-128-2-5p (SEQ ID NO: 44), mmu-miR-434-3p (SEQ ID NO: 45), mmu-miR-496a-5p (SEQ ID NO: 46), mmu-mir-12203 (SEQ ID NO: 47), mmu-miR-12203-3p (SEQ ID NO: 48), mmu-miR-363-5p (SEQ ID NO: 49), mmu-mir-1929 (SEQ ID NO: 50), mmu-miR-7034-5p (SEQ ID NO: 51), mmu-mir-6409 (SEQ ID NO: 52), mmu-miR-5617-5p (SEQ ID NO: 53), mmu-mir-7023 (SEQ ID NO: 54), mmu-miR-2139 (SEQ ID NO: 55), mmu-miR-3102-5p (SEQ ID NO: 56), mmu-miR-871-3p (SEQ ID NO: 57), mmu-miR-8108 (SEQ ID NO: 58), mmu-miR-3073a-3p (SEQ ID NO: 59), mmu-mir-5617 (SEQ ID NO: 60), and mmu-mir-871 (SEQ ID NO: 61).

In another embodiment, the at least one mechanoresponsive miRNA is the human equivalent of an miRNA selected from the group consisting of: mmu-mir-344b (SEQ ID NO: 1), mmu-miR-3060-5p (SEQ ID NO: 2), mmu-miR-344b-3p (SEQ ID NO: 3), mmu-mir-12184 (SEQ ID NO: 4), mmu-mir-293 (SEQ ID NO: 5), mmu-miR-293-3p (SEQ ID NO: 6), mmu-mir-804 (SEQ ID NO: 7), mmu-miR-496a-3p (SEQ ID NO: 8), mmu-miR-1930-5p (SEQ ID NO: 9), mmu-mir-7119 (SEQ ID NO: 10), mmu-miR-1950 (SEQ ID NO: 11), mmu-mir-12201 (SEQ ID NO: 12), mmu-miR-12201-5p (SEQ ID NO: 13), mmu-miR-3109-3p (SEQ ID NO: 14), mmu-miR-124-5p (SEQ ID NO: 15), mmu-miR-7681-5p (SEQ ID NO: 16), mmu-miR-103-1-5p (SEQ ID NO: 17), mmu-miR-219c-3p (SEQ ID NO: 18), mmu-miR-6961-5p (SEQ ID NO: 19), mmu-miR-6244 (SEQ ID NO: 20), mmu-miR-7065-5p (SEQ ID NO: 21), mmu-miR-7675-5p (SEQ ID NO: 22), mmu-miR-7117-3p (SEQ ID NO: 23), mmu-miR-6546-3p (SEQ ID NO: 24), mmu-mir-7675 (SEQ ID NO: 25), mmu-mir-7117 (SEQ ID NO: 26), mmu-mir-6905 (SEQ ID NO: 27), mmu-miR-6905-5p (SEQ ID NO: 28), mmu-miR-5129-3p (SEQ ID NO: 29), mmu-mir-7065 (SEQ ID NO: 30), mmu-mir-1950 (SEQ ID NO: 31), mmu-mir-5129 (SEQ ID NO: 32), and mmu-miR-365-1-5p (SEQ ID NO: 33).

In still another embodiment, the at least one mechanoresponsive miRNA is the human equivalent of an miRNA selected from the group consisting of: mmu-mir-6378 (SEQ ID NO: 34), mmu-miR-6378 (SEQ ID NO: 35), mmu-miR-6403 (SEQ ID NO: 36), mmu-miR-7023-5p (SEQ ID NO: 37), mmu-mir-6539 (SEQ ID NO: 38), mmu-miR-1929-5p (SEQ ID NO: 39), mmu-mir-3470b (SEQ ID NO: 40), mmu-miR-3077-3p (SEQ ID NO: 41), mmu-miR-491-3p (SEQ ID NO: 42), mmu-mir-434 (SEQ ID NO: 43), mmu-miR-128-2-5p (SEQ ID NO: 44), mmu-miR-434-3p (SEQ ID NO: 45), mmu-miR-496a-5p (SEQ ID NO: 46), mmu-mir-12203 (SEQ ID NO: 47), mmu-miR-12203-3p (SEQ ID NO: 48), mmu-miR-363-5p (SEQ ID NO: 49), mmu-mir-1929 (SEQ ID NO: 50), mmu-miR-7034-5p (SEQ ID NO: 51), mmu-mir-6409 (SEQ ID NO: 52), mmu-miR-5617-5p (SEQ ID NO: 53), mmu-mir-7023 (SEQ ID NO: 54), mmu-miR-2139 (SEQ ID NO: 55), mmu-miR-3102-5p (SEQ ID NO: 56), mmu-miR-871-3p (SEQ ID NO: 57), mmu-miR-8108 (SEQ ID NO: 58), mmu-miR-3073a-3p (SEQ ID NO: 59), mmu-mir-5617 (SEQ ID NO: 60), and mmu-mir-871 (SEQ ID NO: 61).

In further embodiments of a method according to the disclosure, “at least one” in the context of the miRNAs may mean one, more than one, or all of each of the above lists of miRNAs.

In one aspect, the disclosure provides a method of promoting regeneration in a subject, comprising administering a composition according to the disclosure to the subject.

In another aspect, the disclosure provides a method of treating a musculoskeletal condition or disorder in a subject, comprising administering a composition according to the disclosure to the subject.

In one aspect, the disclosure provides a method of loading an artificially synthesized exosome with at least one mechanoresponsive miRNA. In one embodiment, the exosome is loaded with the at least one mechanoresponsive miRNA using a method selected from the group consisting of active packaging, electroporation, transfecting mechanosensitive cells, producing hybrid exosome-liposome, and cellular nanoporation.

Other embodiments will become apparent from a review of the ensuing detailed description.

### BRIEF DESCRIPTION OF THE FIGURES

FIG. 1 shows a FlexCell Bioreactor diagram and mechanical strain regimens. Using the FlexCell system, two mechanical strain regimens were created. Their % elongation×time wave functions are shown.

FIGS. 2A to 2I show how mechanical strain influences exosome production. (FIG. 2A) Schematic depicting C2C12 growth following mechanical strain; (FIG. 2B) C2C12 proliferation slows during mechanical strain. Immediately following the mechanical strain regimens, C2C12 cells were collected and counted; (FIG. 2C) C2C12 proliferation increases after mechanical strain. Two-way ANOVA results found cells subjected to the HSSD mechanical strain to be significantly greater than static controls at days 1 and 2, while cells subjected to the LSLD were found to be significantly different at days 1, 2, and 3 (F (6,45)=10.20, n=6/time, p-values found in Tables 2A and 2B, below); (FIG. 2D-2I) Mechanical strain promotes C2C12 exosome production. Isolated exosome supernatants were subjected to BCA total protein assays, CD81 ELISAs, and flow cytometry to determine protein (FIG. 2D), CD81+ (FIG. 2E) and total exosome (FIG. 2I) concentrations (*=p<0.05, **=p<0.01, ***=p<0.001, ****=p<0.0001). (FIG. 2F) size reference beads. (FIG. 2G) unstained exosomes, (FIG. 2H) stained exosomes.

FIG. 3 shows qNano results showing that exosome isolates fall within the 50˜ 150 nm particle size range.

FIGS. 4A-4I show that exosomes from mechanically strained C2C12 cells improve proliferation and differentiation. (FIG. 4A) Schematic depicting naïve C2C12 cell treatment with exosomes; FIGS. 4B and 4C show that exosomes from mechanically strained cells improve C2C12 proliferation (FIG. 4B in graph form, FIG. 4C in quantitative form). Two-way ANOVA results found cells treated with LSLD exosomes to be significantly greater than static at day 3 and 5 F (6, 40)=12.79, n=6/time, p-values found in FIG. 3; FIGS. 4D-4I show that exosomes from mechanically strained cells improve myotube area (FIG. 4D) and myotube nuclear fusion index (FIG. 4E); one-way ANOVAs were performed for all other analysis (*=p<0.05, **=p<0.01. ***=p<0.001, ****=p<0.0001). Staining shown in (FIG. 4F)−exosomes; (FIG. 4G)+static exosomes; (FIG. 4H)+HSSD exosomes; (FIG. 4I)+LSLD exosomes.

FIGS. 5A-5C show (FIG. 5A) that mechanically stimulated cells proliferate faster than static controls; (FIG. 5B) P-values for all comparisons; and (FIG. 5C) 2-way ANOVA with Tukey's HSD results.

FIGS. 6A-6C show (FIG. 6A) that cells treated with exosomes from mechanically strained cells proliferate faster; (FIG. 6B) P-values for all comparisons; (FIG. 6C) 2-way ANOVA with Tukey's HSD results.

FIGS. 7A-7E show that mechanical strain produces unique and differential miRNA within exosomes. (FIG. 7A) the total number of mapped miRNAs per group is listed to the outside, while unique and shared miRNAs are represented within each circle; (FIG. 7B) the heatmap depicts all significant miRNA for each comparison; (FIGS. 7C-7E) volcano plots represent differentially regulated miRNA for each comparison, (FIG. 7C) HSSD vs. static, (FIG. 7D) LSLD vs. static, (FIG. 7E) HSSD vs. LSLD.

FIG. 8 shows Table 2A, which lists the enriched Gene Ontology terms for HSSD vs. static.

FIGS. 9A-9C show miRNA gene ontology. (FIG. 9A) Terminal nodes of enriched Gene Ontology (GO) terms for HSSD vs. static and HSSD vs. LSLD statistically significant miRNA comparisons; blue bars=FDR<0.05 and gray bars=FDR>0.01; (FIG. 9B) GO term categories for the HSSD vs. static comparison; (FIG. 9C) GO term categories for the HSSD vs. LSLD comparison.

### DETAILED DESCRIPTION

Before the present compositions and methods are described, it is to be understood that this disclosure is not limited to particular compositions and methods, and experimental conditions described, as such compositions and methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are incorporated herein by reference in their entirety.

### Definitions

The term “mechanoresponsive”, as used herein, refers to miRNAs that are upregulated and downregulated following mechanical stimuli. The term also refers to miRNAs that are up- or down-regulated in mechanosensitive cells upon exercise.

The term “mechanical stimuli”, as used herein, refers to tension, compression, and/or shear.

The term “mechanosensitive”, as used herein, refers to cells that respond to, or that are known to respond to, various mechanical stimuli. Mechanosensitive cells may include, in various embodiments, isolated muscle cells (e.g., myocytes), stem cells (e.g., mesenchymal stem cells, muscle-derived stem cells, satellite cells), skeletal muscle fiber (e.g., myofibrils), endothelial cells (e.g., blood vessel cells), nerve cells, bone cells (e.g., osteoblasts, osteoclasts, osteocytes), chondrocytes, and cardiomyocytes.

The term “substantially free”, as used herein, refers, in the context of a composition, to a composition being free of cells or other stated substance (other than exosomes), a composition wherein the cells or other stated substance is/are undetectable, or a composition including less than about 1%, less than about 2%, less than about 3%, less than about 4%, less than about 5%, less than about 6%, less than about 7%, less than about 8%, less than about 9%, or less than about 10% cells or other stated substance. For example, the compositions are essentially free of cellular debris, apoptotic bodies, and microvesicles having a diameter less than about 20 nm or greater than about 140 nm.

Furthermore, the term “substantially free”, as used herein, refers, in the context of a composition, to a composition being substantially free of cells, platelets, polypeptides, minerals, blood borne compounds or chemicals, virus, bacteria, fungus, yeast, pathogens, toxins, parasites, and/or the like.

The term “about”, as used herein with respect to any given value, refers to a deviation from that value of up to 10%, either up to 10% greater, or up to 10% less. In additional embodiments, the term refers to a value±5% of that value, ±4% of that value, ±3% of that value, ±2% of that value, or ±1% of that value.

Compositions according to the disclosure are, in some embodiments, administered to a subject in a therapeutically effective amount. The term “therapeutically effective amount”, as used herein, refers to an amount that produces the desired effect for which it is administered. The exact amount will depend on the purpose of the treatment, and will be ascertainable by one skilled in the art using known techniques.

As used herein, the term “subject” refers to an animal, preferably a mammal, more preferably a human. As such, subjects of the disclosure may include, but are not limited to, humans and other primates, such as chimpanzees and other apes and monkey species; farm animals such as cattle, sheep, pigs, goats, and horses; domestic mammals such as dogs and cats; laboratory animals including rodents such as mice, rats, and guinea pigs; birds, including domestic, wild, and game birds such as chickens, turkeys, and other gallinaceous birds, ducks, geese, and the like. In certain embodiments, the subject is a human.

As used herein, the terms “treat”, “treating”, or “treatment” refer to the healing of a bone fracture in a subject in need thereof. The terms include healing of the actual fracture and may additionally or alternatively include ameliorating a symptom associated with the bone fracture, for example, pain, inflammation, reduced mobility, etc.

### Exosomes

Exosomes are nano-sized membrane-bound/membrane-encapsulated vesicles that are released from many cell types into the extracellular space. They are widely distributed in various body fluids. Exosomes are typically about 40 nm to about 100 nm in diameter, but they may have a diameter of about 40 nm to about 140 nm. In one embodiment, exosomes according to the disclosure are about 100 nm in diameter. Exosomes can generally be isolated from, without limitation, whole blood, serum, plasma, urine, saliva, breast milk, cerebrospinal fluid, amniotic fluid, ascitic fluid, and bone marrow of a mammal, as well as from cultured mammalian cells (e.g., immature dendritic cells (wild-type or immortalized), induced and non-induced pluripotent stem cells, fibroblasts, platelets, immune cells, reticulocytes, tumor cells, mesenchymal stem cells, satellite cells, hematopoietic stem cells, pancreatic stem cells, white and beige pre-adipocytes, and the like. However, in embodiments of the compositions according to the instant disclosure, the exosomes are derived from mechanosensitive cells selected from the group consisting of muscle cells, stem cells (e.g., mesenchymal stem cells, muscle-derived stem cells), muscle fiber (e.g., skeletal muscle fiber), endothelial cells (e.g., blood vessel cells), nerve cells, bone cells (e.g., osteoblasts, osteoclasts, osteocytes), chondrocytes, cardiomyocytes, and any other mechanosensitive cells. Cultured cell samples are, in a further embodiment, cultured in cell-appropriate culture media, using exosome-free serum.

Exosomes are advantageous vehicles for miRNAs due to lack of immunoreactivity/immunogenicity and due to their good storage stability. Their lack of immunoreactivity/immunogenicity may make them safe for autologous, allogenic, and xenogenic use. These traits provide exosomes regulatory (FDA approval path) and manufacturing advantages. Finally, the lipid-bilayer nature of exosomes allows for efficient uptake of exosome cargo into recipient cells.

Because exosomes are neither immunoreactive or immunogenic, they could, in one embodiment, hail from an autologous donor that is used for all treatments (e.g., universal donor). For example, a cell line can, in one embodiment, be created from an ideal donor and then maintained for ex vivo exercise and exosome isolation over time.

Exosomes can be isolated from a sample using standard, state-of-the-art methods including, but not limited to, commercially available kits, PEG-based methods, ultrafiltration (for example, ultrafiltration using a tangential fluid filtration (TFF) system (El-Baradie, et al., 2020 Frontiers in Cell Dev Biol 8 (181, pp 1-12), and/or ultracentrifugation/density gradient methods.

Exosomes can be isolated, in one embodiment, by taking advantage of known specific surface markers not present in other vesicles, for example, surface markers such as tetraspanins, e.g., CD9, CD37, CD44, CD53, CD63, CD81, CD82, and CD151.

### Artificial Exosomes

In certain embodiments of the compositions and methods according to the disclosure, the exosomes are artificially synthesized as engineered lipid nanoparticles. The artificial exosomes of certain compositions and methods of the disclosure are engineered for stability, circulating ability (long t_{1/2}), targeting ability, and biocompatibility (i.e., minimized cytotoxicity and immunogenicity). In one embodiment, an artificial exosome is synthesized using standard, state-of-the-art nanofluidic technology. A nanofluidic device is used to make uniform lipid nanoparticles. The composition of lipid nanoparticles is then engineered to be biocompatible (e.g., non-inflammatory) and to encapsulate mechanoresponsive miRNAs. The lipid composition is based on the state of the art of the field.

In one embodiment, artificial exosomes according to the disclosure are engineered to be about 100 nm in diameter. In another embodiment, a composition according to the disclosure comprises exosomes that are substantially consistent in size.

miRNAs

mRNAs and microRNAs (miRNAs) have been identified in exosomes, which can be taken up by recipient cells and subsequently modulate the same. An active sorting mechanism of exosomal miRNAs may be indicated, since the miRNA profiles of exosomes may differ from those of the parent cells.

microRNAs are small, single-stranded non-coding ribonucleic acid molecules about 18 to about 28 nucleotides long, with an average length of about 22 nucleotides, They regulate gene expression at the posttranscriptional level by either changing mRNA stability and degrading them or by inhibiting protein translation (Fabian, et al., 2010 Annu Rev Biochem 79:351-379). miRNAs thus modulate the activity of numerous target genes and are implicated in fundamental physiological processes, Due to their robust biological output, these regulatory RNAs (˜22 bp) may offer a relatively simple, inexpensive, and scalable way to guide cellular activities.

Precursor miRNAs (pre-miRs or “mirs”) are processed by ribonuclease III enzymes, for example, Dicer or Drosha, into mature miRNAs (or “miRs”) after a series of processing steps.

A subset of exosomal miRNAs increases in abundance following exercise, suggesting an exercise-induced release of exosomes enriched in specific miRNAs. The exosomes disclosed herein comprise at least one mechanoresponsive miRNA. The murine miRNAs are listed in Table 1, further below. In certain embodiments, the exosomes can comprise 1, more than one, or all of the human equivalents of the mechanoresponsive miRNAs listed in Table 1 under “HSSD” (high strain short duration). In other embodiments, the exosomes can comprise 1, more than one, or all of the human equivalents of the mechanoresponsive miRNAs listed in Table 1 under “LSLD” (low strain long duration). And in still other embodiments, the exosomes can comprise 1 each or more than one each of the human equivalents of the mechanoresponsive miRNAs listed in Table 1 under “HSSD” and “LSLD”. Three human equivalent miRNAs are disclosed herein (but are not included in Table 1): hsa-miR-124-5p (SEQ ID NO: 62), hsa-miR-491-3p (SEQ ID NO: 63), and hsa-miR-128-2-5p (SEQ ID NO: 64).

In some embodiments, mechanoresponsive miRNAs can be identified and/or characterized using bioinformatics and/or an assay selected from the group consisting of qPCR, miRNA array, and RNA-seq.

### Exercise

Exercise is generally defined as an activity requiring physical effort. It may be carried out to sustain or improve health and fitness. The term “exercise” may encompass endurance exercise, high-intensity interval training, resistance exercise, and the like, as well as combinations thereof. In one embodiment, a mammal exercises, for example, on a treadmill.

In certain embodiments of compositions and methods according to the disclosure, exosomes are isolated from a cell sample (i.e., in vitro) after ex vivo exercise. This ex vivo exercise is achieved by a bioreactor creating mechanical tension on cells in the sample. The mechanical bioreactor, in these embodiments, simulates endogenous exercise. For muscle cells or fibers and the like, mechanical loading may, for example, comprise axial loading and/or uniaxial tension. For chondrocytes and the like, for example, mechanical loading may comprise compressive and shear loading.

Exercise is, in some embodiments, referred to herein as low intensity or high intensity. Low intensity corresponds to low strain for long duration (LSLD), while high intensity corresponds to high strain for short duration (HSSD). An LSLD regimen, in one embodiment, comprises subjecting cells to 0-15% strain (% elongation) at 0.5 Hz for 24 hrs. an HSSD regimen, in one embodiment, comprises subjecting cells to 12-22% strain (% elongation) at 1 Hz for 10 minutes, followed by 50 minutes rest at 0% strain, for 24 hours. These regimens are to be seen as representative, but not limiting, of low intensity and high intensity exercise. The bioreactor used may, for example, define or limit the low vs. high intensity training exercise. Thus, a different bioreactor may allow for different intensity parameters; these parameters are likewise contemplated for the compositions and methods according to the disclosure.

The biological sample or cell sample is obtained immediately after (within 60 seconds, within 5 minutes, within 10 minutes, within 15 minutes, within 30 minutes, within 1 hour, within 2 hours, within 3 hours, or within 4 hours of) completion of exercise regimen, and the exosomes are subsequently isolated therefrom.

### Compositions

Exosomes obtained according to the disclosure may be formulated for therapeutic use by combination with a pharmaceutically or physiologically acceptable carrier. The terms “pharmaceutically acceptable” or “physiologically acceptable”, as used herein, refer to carriers that are acceptable for use in the pharmaceutical and veterinary arts. The carrier may be selected with a view to its intended use and/or its intended administration method.

In one embodiment, the exosomes are formulated as a suspension in a medical-grade, physiologically acceptable carrier, such as an aqueous solution in sterile and pyrogen-free form, optionally, buffered or made isotonic. The carrier may be distilled water (DNase- and RNase-free), a carbohydrate-containing solution (e.g., sucrose or dextrose) or a saline solution comprising sodium chloride and optionally buffered. Suitable saline solutions may include varying concentrations of sodium chloride, for example, normal saline (0.9%), half-normal saline (0.45%), quarter-normal saline (0.22%), and solutions comprising greater amounts of sodium chloride (e.g., 3%-7%, or greater). Saline solutions may optionally include additional components, e.g., carbohydrates such as dextrose and the like. Examples of saline solutions including additional components, include Ringer's solution, e.g., lactated or acetated Ringer's solution, phosphate buffered saline (PBS), TRIS (hydroxymethyl) aminomethane hydroxymethyl) aminomethane)-buffered saline (TBS), Hank's balanced salt solution (HBSS), Earle's balanced solution (EBSS), standard saline citrate (SSC), HEPES-buffered saline (HBS), and Gey's balanced salt solution (GBSS).

(Pharmaceutical) compositions in accordance with the disclosure are administered with suitable excipients, and/or other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell, et al., “Compendium of excipients for parenteral formulations”, PDA (1998), J Pharm Sci Technol 52:238-311.

In certain embodiments, the excipient is simply water, and in one embodiment, pharmaceutical grade water. In other embodiments, the excipient is a buffer, and in one embodiment, the buffer is pharmaceutically acceptable. Buffers may also include, without limitation, saline, glycine, histidine, glutamate, succinate, phosphate, acetate, aspartate, or combinations of any two or more buffers.

In other embodiments, a hydrogel carrier is included in the composition. In additional embodiments, the hydrogel is viscous, yet still flowable, and in other embodiments, the hydrogel is solid, semi-solid, gelatinous or of any density in between. Accordingly, in various embodiments and without limitation, the hydrogel is natural a substance comprised of collagen, gelatin, gluten, elastin, albumin, chitin, hyaluronic acid, cellulose, dextran, pectin, heparin, agarose, fibrin, alginate, carboxymethylcellulose, Matrige™ (a hydrogel formed by a solubilized basement membrane preparation extracted from the Engelbreth-Holm-Swarm (EHS) mouse sarcoma), hydrogel organogel, or mixtures and/or combinations thereof.

In another embodiment, microparticles are synthesized from one or more polymers, one or more copolymers, one or more block polymers (including di-block polymers, tri-block polymers, and/or higher multi-block polymers), as well as combinations thereof. Useful polymers include, but are not limited to, polylactic acid (PLA), polyglycolic acid (PGA), poly(.epsilon.-caprolactone) (PCL), poly(ethylene glycol) diacrylate (PEGDA), poly(ethylene glycol) dimethacrylate (PEGDMA), SU-8, poly(methyl methacrylate), poly(lactide-co-glycolide), poly-caprolactone, and elatin/caprolactone, collagen-GAG, collagen, fibrin, poly(anhydrides), poly(hydroxy acids), poly(ortho esters), poly(propylfumerates), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polyethylene, polycarbonates, poly(ethylene oxide), polydioxanone, “pseudo-polyamino acid” polymer based on tyrosine, tyrosine-derived polycarbonate poly(DTE-co-DT carbonate), tyrosine-derived polyarylate, polyanhydride, trimethylene carbonate, poly(.beta.-hydroxybutyrate), poly(g-ethyl glutamate), poly(DTH iminocarbonate), poly(bisphenol A iminocarbonate), poly(ortho ester), polycyanoacrylate, and polyphosphazene, poly(lactide-co-glycolide) (PLGA), poly(DL-lactide-co-epsilon.-caprolactone) (DLPLCL), a modified polysaccharide (cellulose, chitin, dextran) a modified protein, casein- and soy-based biodegradable thermoplastics, collagen, polyhydroxybutyrate (PHB), multiblock copolymers of poly(ethylene oxide) (PEO) and poly(butylene terephthalate) (PBT), polyrotaxanes. In other embodiments, the microparticles are formed from one or more phospholipids. 2-methacryloyloxyethyl phosphorylcholine (MPC), one or more cationic polymers (poly(a-[4-aminobutyl]-L-glycolic acid), or one or more silicone-urethane copolymers. In still other embodiments, microparticles are formed from co-polymers of any of the above, mixtures of the above, and/or adducts of the above. Again, the worker of ordinary skill in the art will appreciate that any pharmaceutical grade matrix is amenable for use in a composition of the disclosure.

Injectable compositions may include dosage forms for intravenous, subcutaneous, percutaneous, intramuscular injections, drip infusions, etc. These injectable preparations may be prepared by methods publicly known.

Compositions according to the disclosure are, in one embodiment, stored in liquid form. In another embodiment, compositions according to the disclosure are stored in lyophilized form. In still another embodiment, compositions according to the disclosure can be freeze-dried with lyoprotectants, for example PVP+trehalose (El-Baradie, et al., 2020 Frontiers in Cell Dev Biol 8 (181, pp 1-12)). In some embodiments, liquid compositions according to the disclosure are stably stored as at −80° C. for at least about 6 months. In some embodiments, liquid compositions according to the disclosure are stably stored at −20° C. for at least about 3 months and up to about 6 months. In other embodiments, lyophilized compositions according to the disclosure are stably stored at room temperature.

### Administration

Certain aspects of the present disclosure include administering a composition comprising exosomes comprising mechanoresponsive miRNAs to a subject. In practicing the methods and uses according to certain embodiments of the disclosure, a composition comprising exosomes comprising mechanoresponsive miRNAs is administered to a subject.

In certain embodiments, a composition according to the disclosure is administered locally. The term “locally”, as used herein, may refer to an injury or surgery site, or adjacent to an injury or surgery site.

Modes of administration may include, but are not limited to injection (e.g., percutaneously, subcutaneously, intravenously, or intramuscularly, intrathecally). In certain embodiments, the mode of administration is surgical implantation (for example, of a hydrogel/matrix as described herein).

A composition according to the disclosure can, in certain embodiments, be delivered subcutaneously or percutaneously with a standard needle and syringe. In addition, a pen delivery device may, in one embodiment, be employed to administer a composition of the present disclosure.

In certain embodiments, a composition according to the disclosure can be delivered in a controlled release system. In one embodiment, a pump may be used. In another embodiment, polymeric materials can be used. In yet another embodiment, a controlled release system can be placed in proximity of the composition's target, thus requiring only a fraction of the systemic dose.

In certain embodiments, the frequency and the duration of the administration can be adjusted. In certain embodiments, the composition can be administered as a single dose of exosomes. In other embodiments, the composition can be administered as an initial dose, followed by administration of a second or a plurality of subsequent doses of the exosomes comprising at least one mechanoresponsive miRNA in an amount that can be approximately the same or less than that of the initial dose, wherein the subsequent doses are separated by at least one week, at least 2 weeks, at least 3 weeks, at least one month, at least two months, at least three months, or longer, based on a lack of adequate progression of desired effect. In certain embodiments, a lack of adequate progression of desired effect comprises no reduction in pain, minimal reduction in pain, no or minimal increase in stability, no or minimal increase in mobility, no or low (bone) mineralization on X-ray, no or low reduction in inflammation, and/or no or low increase in strength. A clinician would be able to change the frequency and duration of treatment on a per patient basis based on their diagnosis and unique condition.

### Uses

Compositions according to the disclosure are, in some embodiments, for use in promoting regeneration in a subjection. Promoting regeneration comprises promoting proliferation and/or cellular differentiation. In a further embodiment, compositions according to the disclosure are for use in promoting muscle repair and/or promoting muscle regeneration. In still a further embodiment, compositions according to the disclosure are for use in promoting bone repair. In one embodiment, compositions according to the disclosure are for use in replacing orthobiologics in any situation in which orthobiologics are being used. In another embodiment, compositions according to the disclosure are for use in replacing orthobiologics (for example, platelet-rich plasma (PRP), bone marrow aspirate) in the treatment of a musculoskeletal condition or disorder. In yet another embodiment, compositions according to the disclosure are for use in use in orthopedic recovery post-surgery. In a further embodiment, compositions according to the disclosure are for use in reducing a pro-inflammatory state. In still a further embodiment, compositions according to the disclosure are for use in improving the age state of a person through cellular rejuvenation.

Compositions according to the disclosure are, in further embodiments, for use in the treatment of a musculoskeletal condition or disorder. In specific embodiments, the musculoskeletal condition or disorder is selected from the group consisting of, but not limited to, atrophy, sarcopenia, osteoarthritis, osteoporosis, injury, and age-related frailty.

In one embodiment, a subject may be unable to exercise as required or to exercise at all; a (mechanosensitive) cell sample could be obtained from the patient and exercised ex vivo, exosomes could be isolated from the cells in the sample, and a composition comprising the isolated exosomes could then be administered to the subject. In another embodiment, the cell sample hails from a universal autologous donor.

In another embodiment, a composition comprising mechanoresponsive miRNAs as disclosed herein is characterized with a view to personalizing an exercise program for a subject to maximize the promotion of regeneration in the subject. In such an embodiment, the injury or disease is characterized, and a composition is prepared comprising exosomes that comprise the mechanoresponsive miRNAs that affect the regulatory processes of relevance to the subject. Alternatively, mechanoresponsive miRNAs are identified and characterized in exosomes released after varying types and intensities of exercise. An exercise regimen is then tailored to a subject based on the mechanoresponsive miRNAs they “need” (i.e., those mechanoresponsive miRNAs that affect the regulatory processes of relevance to the subject).

### Combination Therapies

In some embodiments, a composition according to the disclosure is administered to a subject in combination with another therapeutic agent or therapy.

As used herein, the term “in combination with” means that at least one additional therapeutic agent/therapy may be administered prior to, concurrent with, or after the administration of the composition comprising exosomes comprising mechanoresponsive miRNAs.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Exosome Preparation and miRNA Characterization

### 1.1 Methods, Cell Culture

C2C12 myoblasts (ATCC, catalog #CRL-1722) were cultured in growth media containing Dulbecco's Modified Eagle Medium (DMEM; Gibco, catalog #11995040), 10% Fetal Bovine Serum (FBS; Gibco, catalog #10437028), and 1% penicillin/streptomycin (P/S; Gibco, catalog #15140148) at 37° C. in humidified air containing 5% CO2. Cells were maintained in tissue culture treated T75 flasks (Corning, catalog #10-126-11) and not passaged more than eight times.

### 1.2 Methods, Mechanical Strain

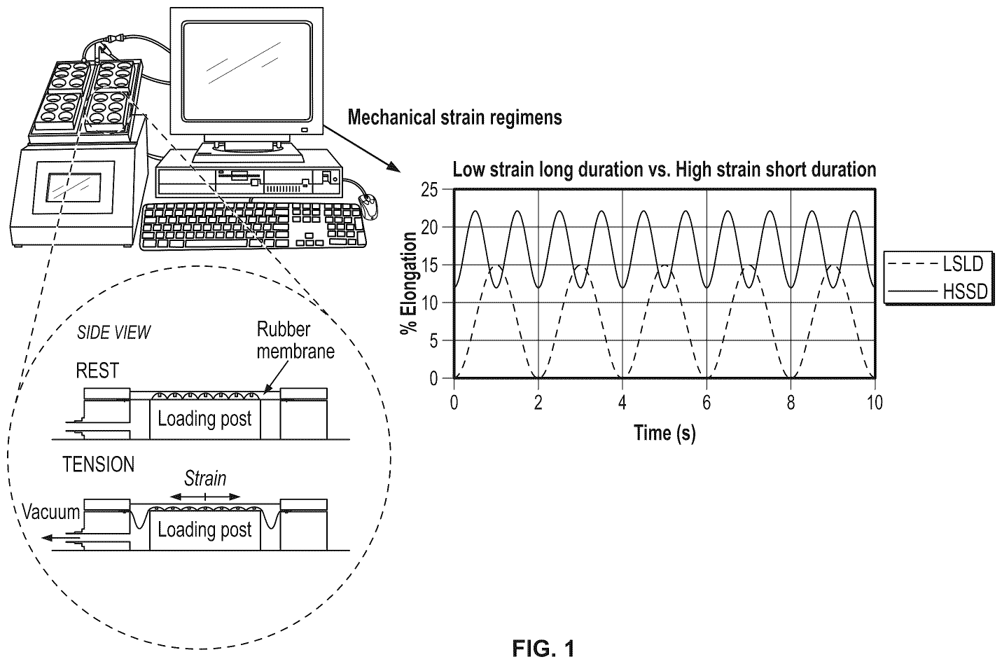

Using a commercially available bioreactor (FlexCell International, catalog #FX-5000TT), 100,000 C2C12 cells per well were plated onto 6-well BioFlex culture plates (FlexCell International, catalog #BF-3001U). Two cyclic tension regimens were created. The first regimen subjected cells to a low strain for a long duration (LSLD) of 0-15% strain at 0.5 Hz for 24 hours. The second subjected cells to a high strain for a short duration (HSSD) of 12-22% strain at 1 Hz for 10 minutes followed by 50 minutes of rest at 0% strain for 24 hours. Static (“unexercised”) groups were plated in parallel to the LSLD and HSSD (“exercised”) groups (FIG. 1).

### 1.3 Methods, Exosome Isolation

Media containing exosome-depleted FBS (Gibco, catalog #A2720803) was added and incubated with the cells for the 24-hour “exercise” regimen. The media was collected and centrifuged at 1,500×g for 10 minutes followed by a second centrifugation at 3,000×g for 10 minutes before being centrifuged at 4,000×g for 10 minutes using 100 kDa concentrating columns (Amicon, catalog #UFC910024). The concentrated media was then incubated with the Invitrogen Total Exosome Isolation Reagent (Invitrogen, catalog #4478359) overnight at 4° C. before being centrifuged at 10,000×g for 1 hour at 4° C. Exosome pellets were resuspended in 500 μL of 1×0.02 μm filtered PBS and stored at −80° C.

### 1.4 Methods, C2C12 Post-Exercise Regimen Proliferation

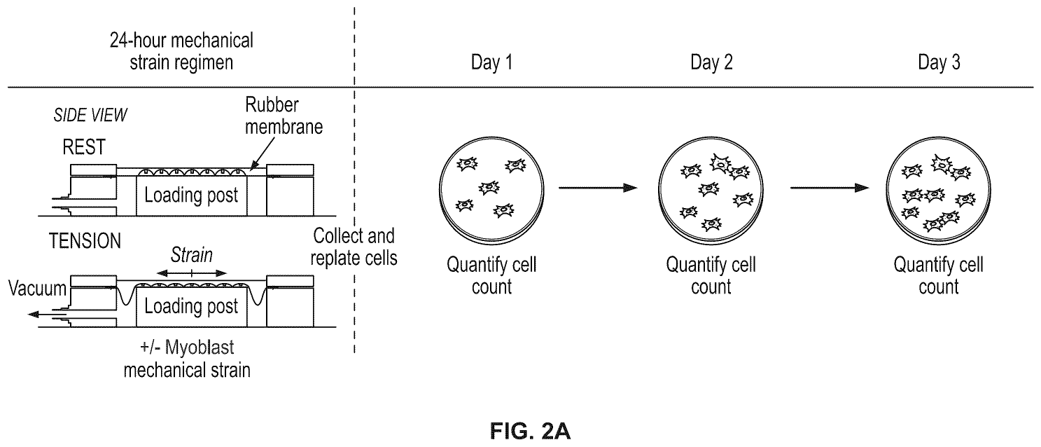

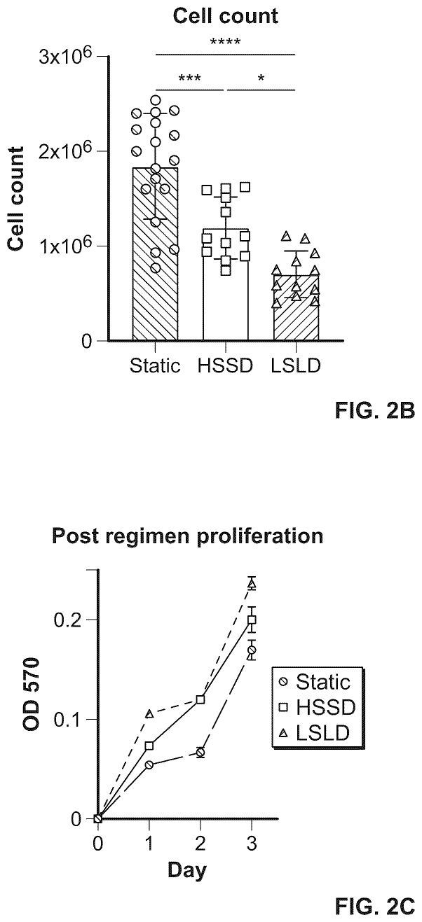

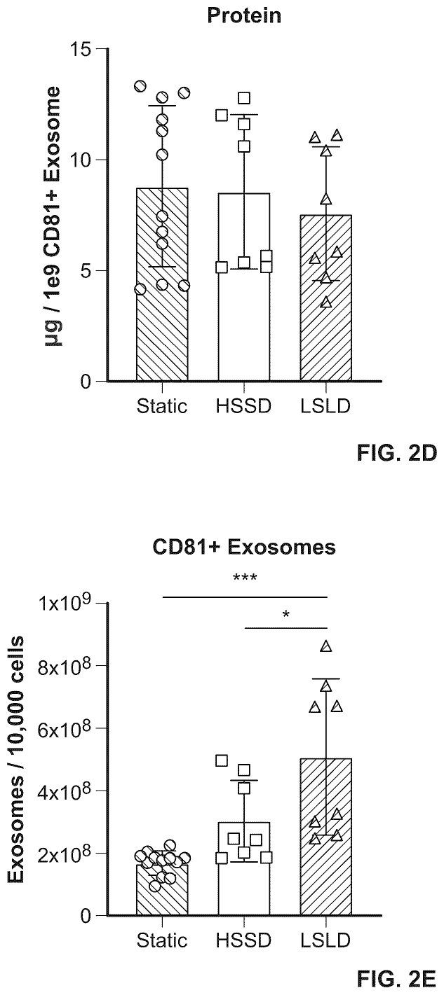

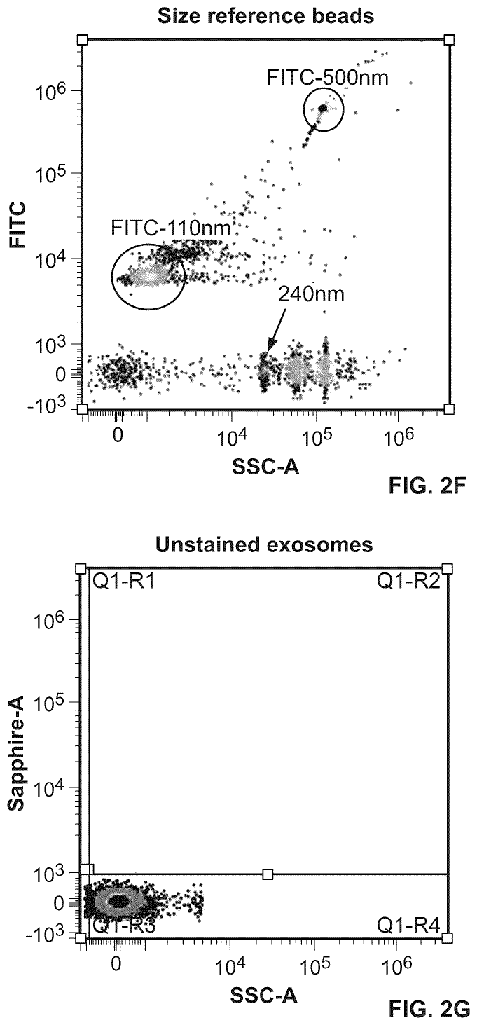

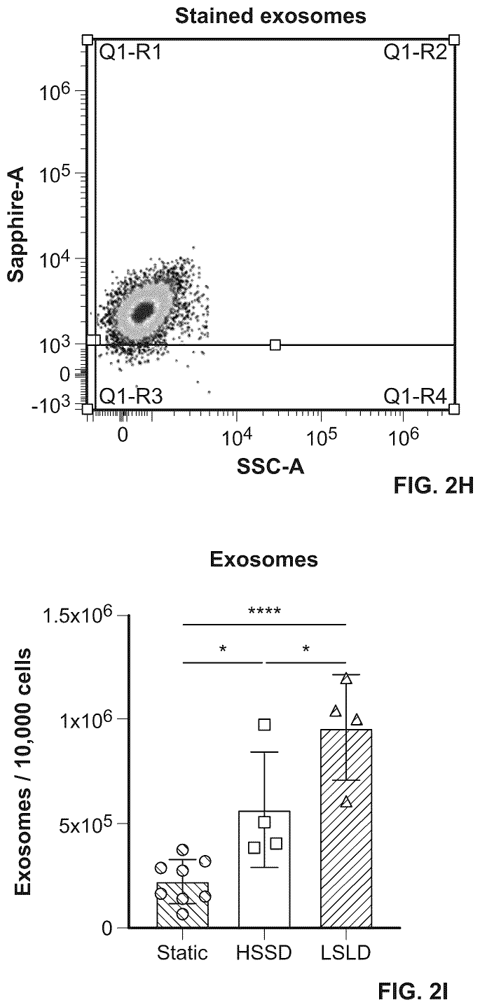

Following the mechanical strain regimens, the cells were collected, counted, and 5,000 C2C12 cells from each group were replated into separate wells of a 24-well plate (Genesee, catalog #25-107), After adhering overnight, the cells were treated with the PrestoBlue Cell Viability Reagent according to the manufacturer's instructions at days 1, 2, and 3 (FIG. 2A).

### 1.5 Methods, Exosome and Exosome Protein Content Quantification

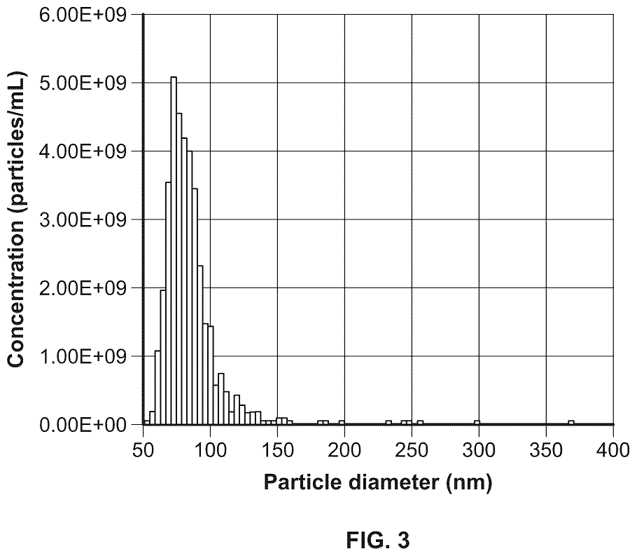

Two methods were used to quantify exosome numbers in the conditioned media from each experimental condition. Exosome tetraspanin marker CD81 was quantified using System Bioscience's ExoELISA-ULTRA Complete CD81 Detection according to the manufacturer's instructions (SBI, catalog #EXEL-ULTRA-CD81-1). Aggregate exosome concentrations were quantified using System Biosciences ExoFlow-ONE EV Labeling Kit (SBI, catalog #EXOF400A-1) on the Cytek Northern Lights flow cytometer (Cylek, catalog #NL-3000). Methodologies followed SBI's staining protocol and a previously published protocol for optimized quantification using the Cytek Northern Lights instrument (Coughlan, et al., 2020 Curr Protoc Cell Biol 88 (1). https://pubmed.ncbi.nlm.nih.gov/32633898/). To ensure accurate measurements were made, a serial dilution of aggregate exosome sample was made and quantified for each run. Sample results were only quantified if the run's corresponding serial dilution curve had an R2 exceeding 0.9. Exosome protein concentrations from each group were determined using the Pierce Microplate Bicinchonicic Acid Assay according to manufacturer's instructions (BCA; Thermo, catalog #23252). The Izon qNano Gold TRPS instrument was also used to confirm isolation of exosome sized extracellular vesicles (FIG. 3).

### 1.6 Methods, Naïve C2C12 Treatment with Exosomes

Using normal growth media 5,000 C2C12 cells were plated into separate wells of a 24-well plate. After adhering overnight, the cells were washed and treated with PBS or 10 μg/ml of static, HSSD, or LSLD exosomes on days 1 and 3. Growth media containing exosome-depleted FBS was used for all treatments. Following manufacturer's instruction, the PrestoBlue Cell Viability Reagent was used to determine cell proliferation at day 1 (prior to treatment), day 3 (48 hours after the first treatment), and day 5 (48 hours after the second treatment).

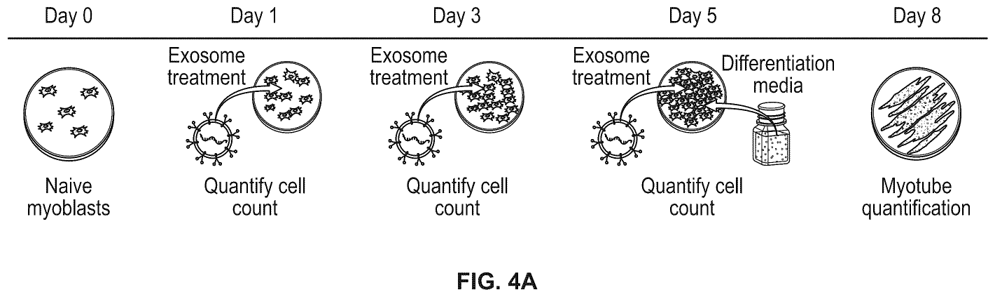

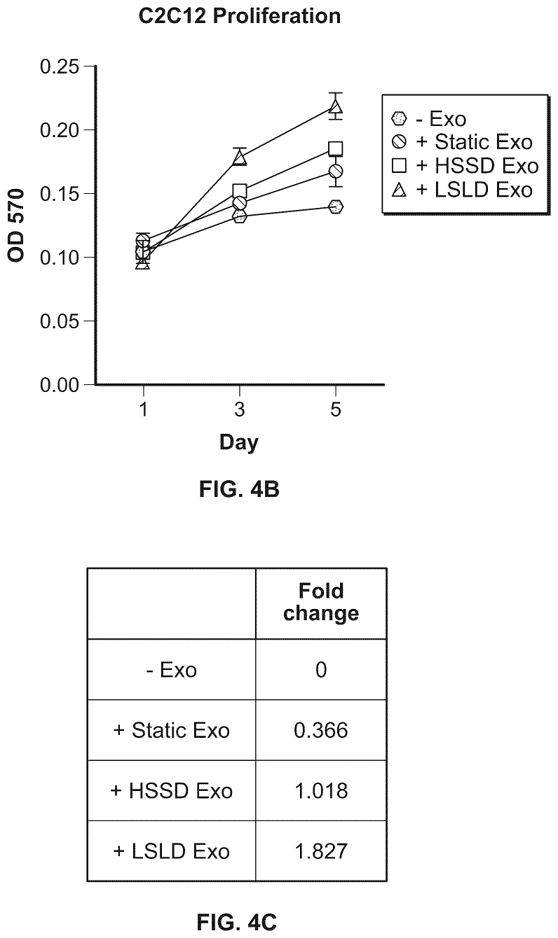

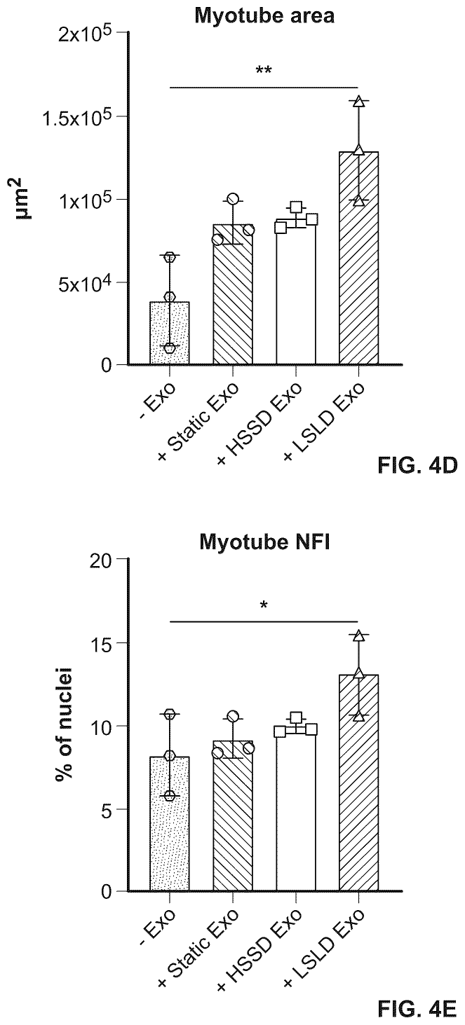

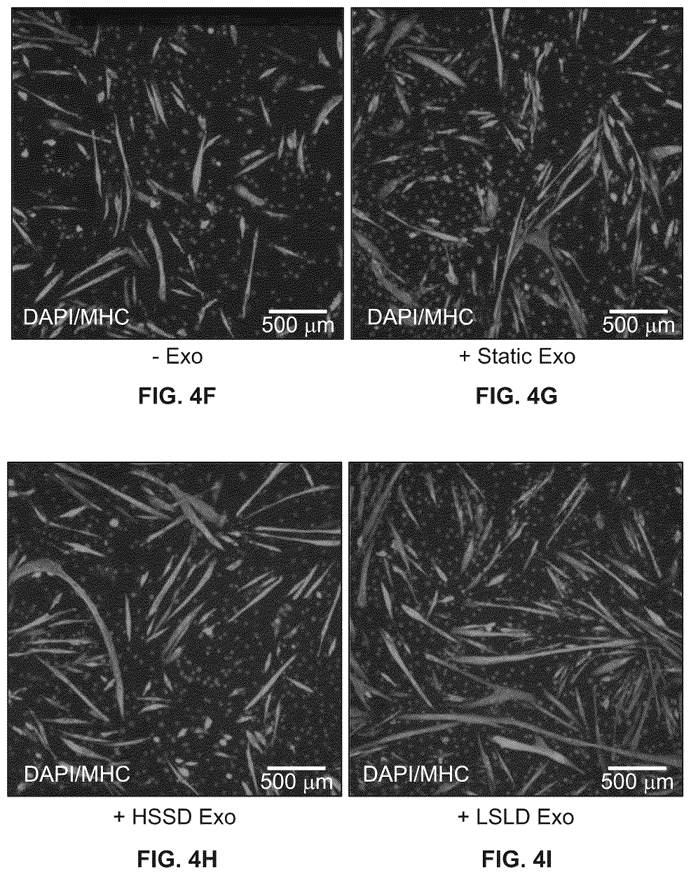

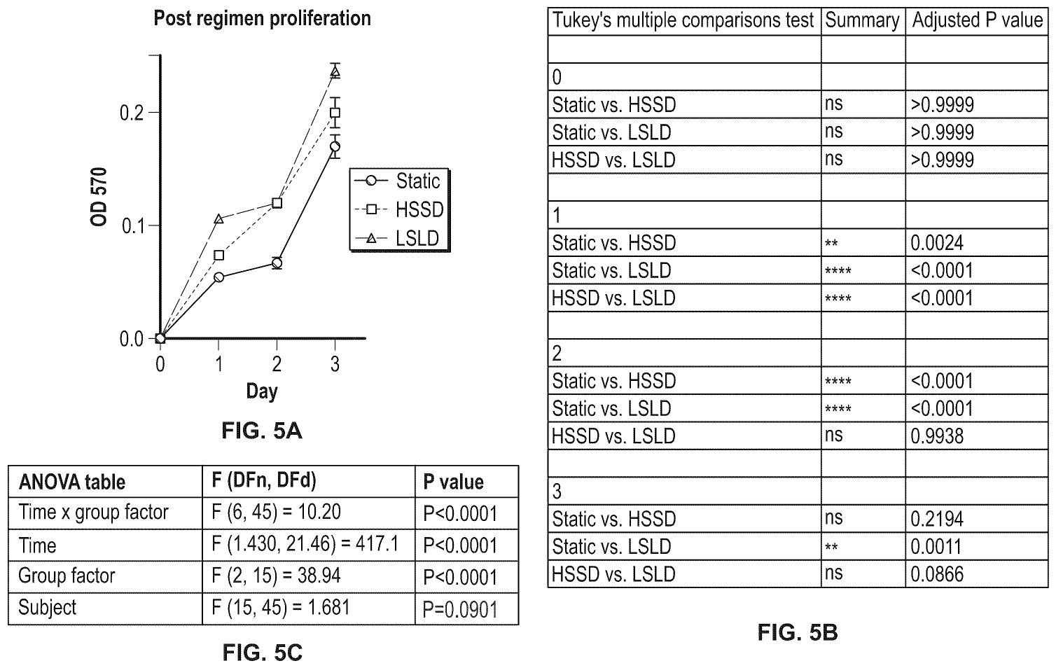

At day 5, the cells were treated with differentiation media supplemented with PBS or 10 μg/mL of static, HSSD, or LSLD exosomes. C2C12 differentiation media contained 2% exosome-depleted FBS. C2C12 cells were subsequently cultured for 3 days (FIG. 4A).

### 1.7 Methods, Immunofluorescence Staining

Cells were washed with PBS, fixed using cold 4% PFA (Alfa Aeser, Catalog #J19943K2) for 15 minutes, and washed three more times. The cells were then blocked using 10% donkey serum (DS; Jackson Immuno, catalog #017000121) and 0.3% Triton X-100 (Fisher, catalog #BP151-500) in PBS for 1 hour followed by a 3-hour incubation at room temperature with the myosin heavy chain antibody (MHC: R&D Systems, Catalog #MAB4470). 10 μg/mL of MHC was suspended in dilution buffer containing 1×PBS with 1% DS, 1% bovine serum albumin (BSA; Sigma, catalog #A9647-100G), and 0.3% Triton X-100. The cells were then washed three times using 1×PBS with 0.1% BSA and then incubated with 1:400 diluted Alexa Flour 594 (Invitrogen, catalog #A21203) for 1 hour at room temperature. The cells were washed three more times and incubated with 1 μg/ml of DAPI (Sigma, catalog #D9542-10 MG) for 10 minutes before being washed.

### 1.8 Methods, Myotube Quantification

Cells were imaged using the Nikon Eclipse Ti-E microscope. Three wells were imaged per group and within each well sixteen 10× images were taken in a 4×4 automated grid pattern. The opensource MyoCount program was then used to process the images and determine the myotube area and the nuclear fusion index of each image (Murphy, et al., 2019 Wellcome Open Res 4 https://pubmed.ncbi.nlm.nih.gov/30906880/).

### 1.9 Methods, miRNA Next Generation Sequencing

Three exosome replicates from each group (static, HSSD, and LSLD) were sent to System Biosciences for exosomal RNA sequencing. The miRNA samples were isolated and sequenced using SBI's protocols and, upon sequencing, the results were analyzed using the Exosome Small RNA-seq Analysis Kit on the cloud-based Maverix Analytic Platform. Data analysis was visualized and downloaded using the University of California Santa Cruz Banana Slug Analytics Platform.

### 1.10 Methods, miRNA Gene Ontology

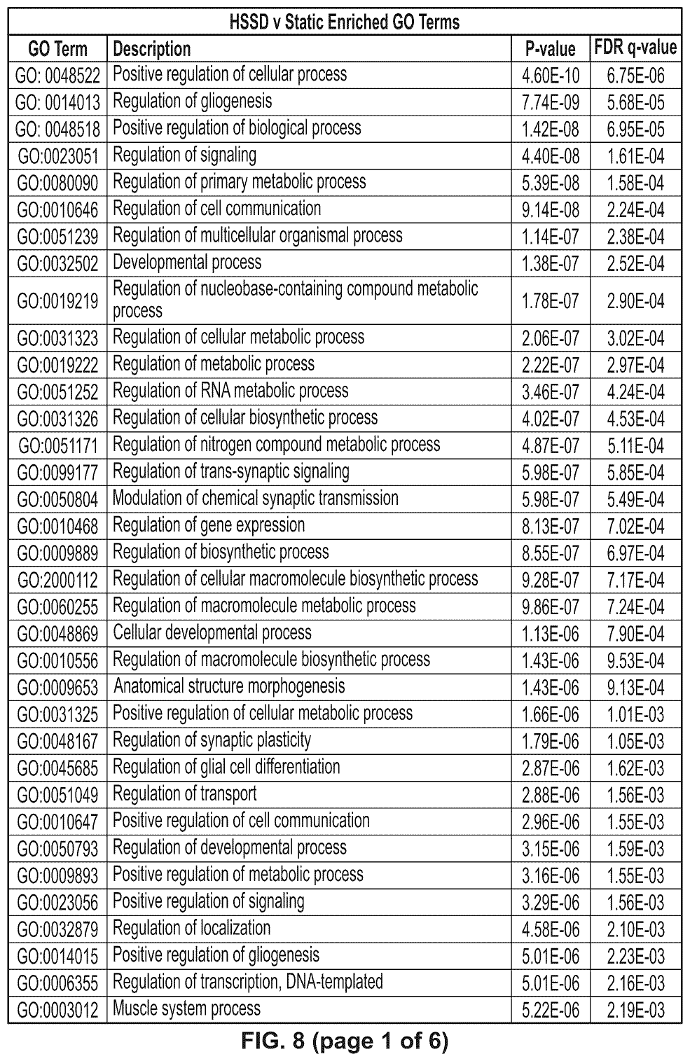

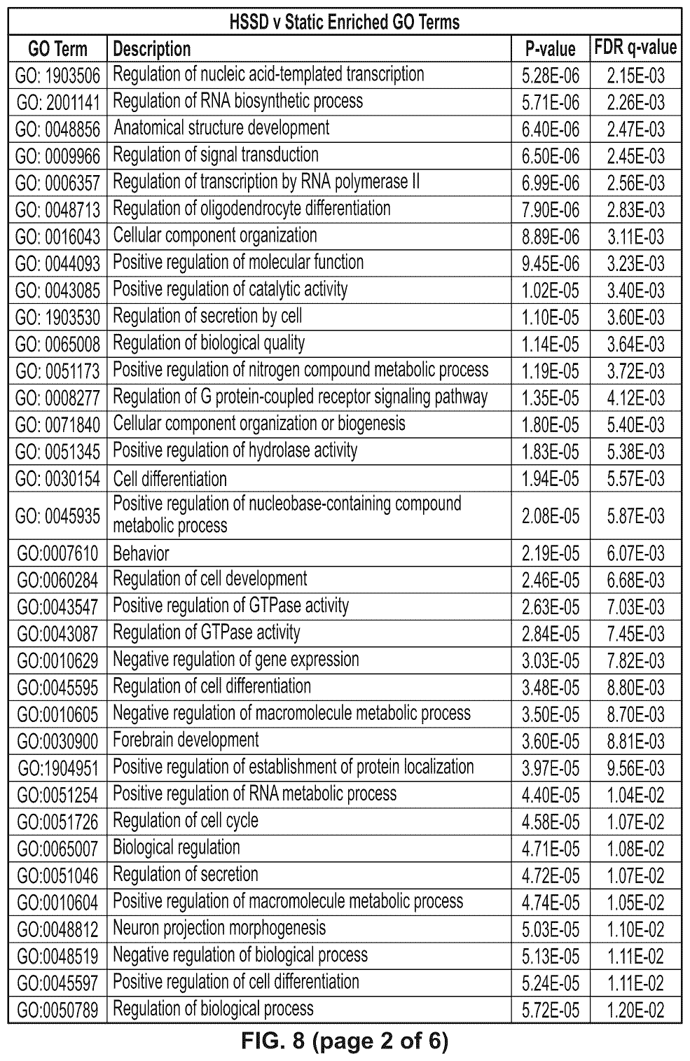

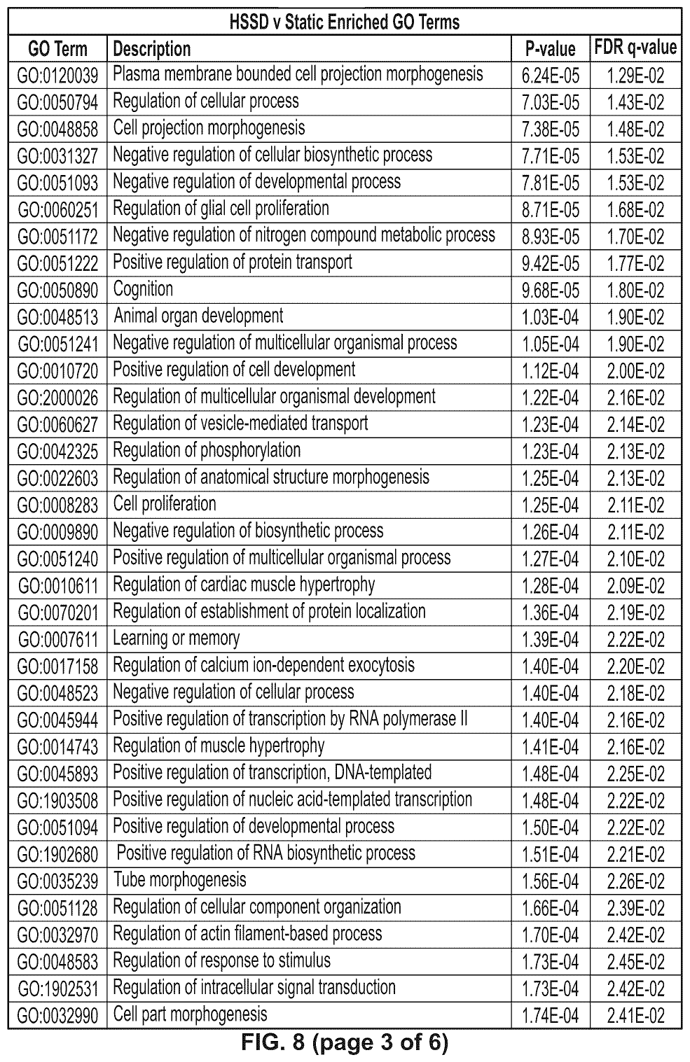

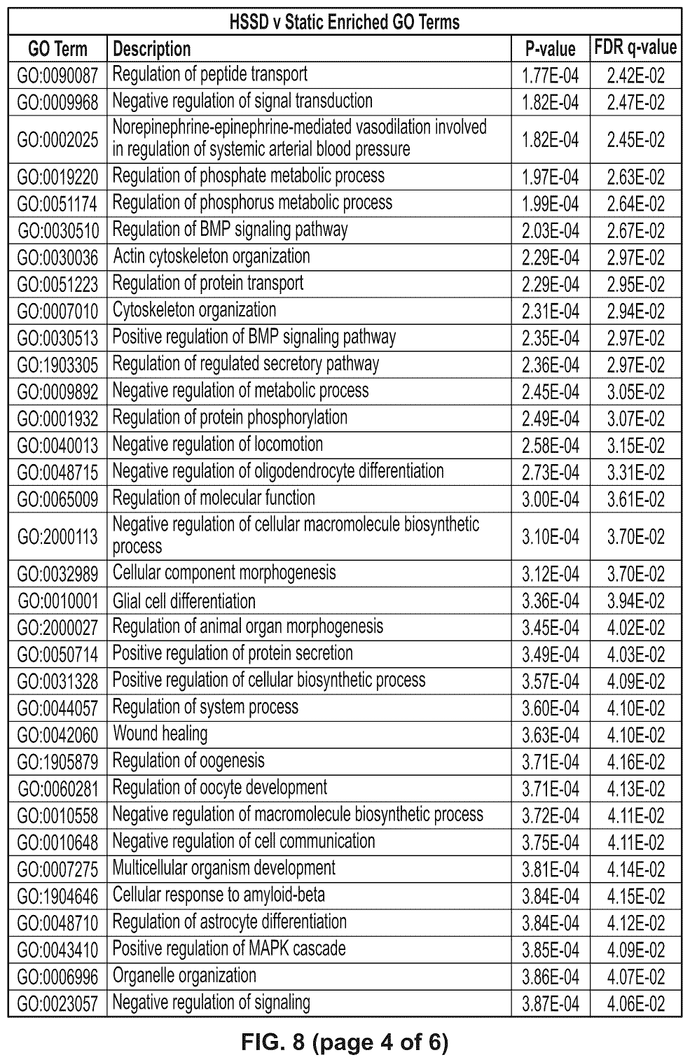

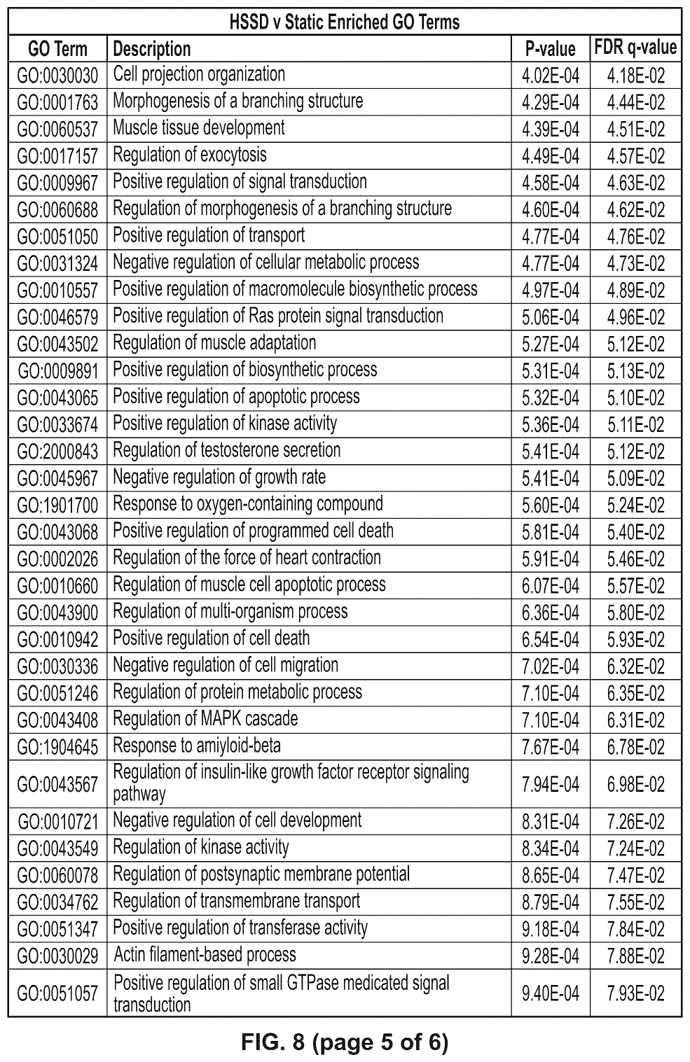

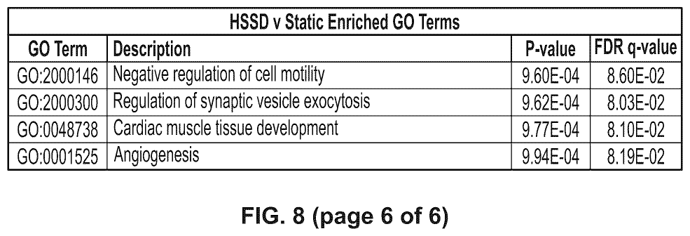

Significantly up-/down-regulated miRNAs were determined by Banana Slug Genomics and analyzed independently for gene targets using mirDB (Chen and Wang, 2020 Nucleic Acids Res 48, Liu and Wang, 2019 Genome Biol 20 (1)). A threshold of 95% target predication score was used for downstream analysis of miRNA gene targets. Gene targets with a target prediction score of greater than or equal to 95% were then analyzed for enriched Gene Ontology (GO) terms using GOrilla with Mus musculus as the target organism (Eden, et al., 2009 BMC Bioinformatics 10 (1), Eden, et al., 2007 PLOS Comput Biol 3 (3)). Gene targets were analyzed as two unranked lists of gene sets, with the Mus musculus proteome as the background gene set. Enriched GO terms are shown in Tables 2A (FIG. 8) and 2B, below. A terminal node analysis was then performed on the enriched GO terms in R Studio using the Bioconductor GO.db package, and the most specific enriched GO terms (terminal nodes) with FDR<0.1 were reported.

To categorize the enriched GO terms, four reviewers were prompted with seven umbrella categories (development, signaling, metabolism, muscle, neural, inflammation, and transcriptional regulation) and asked to assign one category to each GO term. The reviewers were blinded to one another's responses and the category selected by the majority of the reviewers was assigned to each GO term (Tables 3A and 3B, below).

### 1.11 Methods, Statistics

All statistical comparisons and figures were generated using the PRISM 9 analysis software. All mean values and standard deviations were determined from at least three replicates. Where present, individual data points represent one replicate. One-way ANOVAs were completed to test for significant difference between multiple comparisons, while two-way ANOVAs were run to compare multiple groups over time. Tukey's honestly significant difference (HSD) post-hoc testing was completed in both scenarios. Values with a p<0.05 were considered to be statistically significant.

### Results

### Mechanical Strain Influences Exosome Production

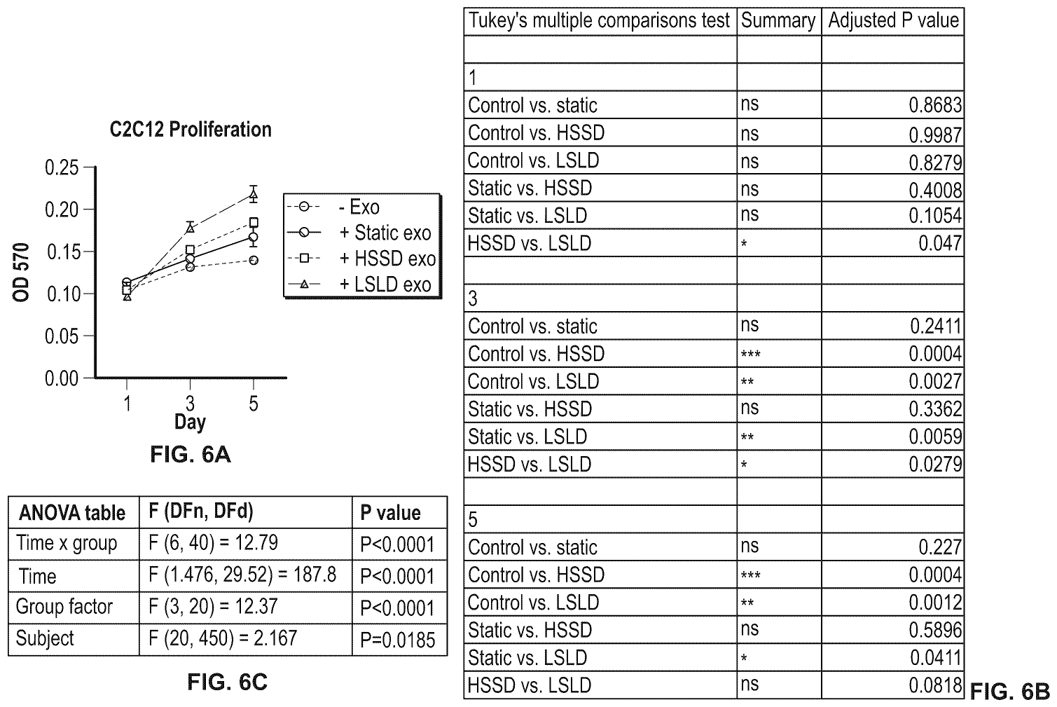

Following 24 hours of mechanical strain (FIG. 1), C2C12 cell counts from both HSSD and LSLD groups were significantly lower than the static group at the completion of their respective regimens (FIG. 2B). This led to the question of whether mechanical strain impacted C2C12 proliferation. Using the PrestoBlue Cell Viability Reagent to colorimetrically determine the relative number of cells for 3 days following the completion of loading and replating at equal densities, cell number was found to be different in both a time and group component with a two-way ANOVA analysis (F_{6,45}=10.20, (FIG. 2C), time and group comparisons found in FIG. 6). With respect to the different loading groups, after replating at equal densities, cells from the LSLD regimen were increased when compared to the static at days 1, 2, and 3 (p<0.0001, p<0.0001, and p<0.0011, respectively). Similarly, the cell counts subjected to the HSSD regimen were also higher than the static at days 1 and 2 (p=0.0024 and p<0.0001, respectively). When comparing the LSLD and HSSD groups, it was found that the LSLD cell count was only greater at day 1 (p<0.0001). Within each group, cell populations were significantly greater at each day when compared to the previous day (p<0.0001).

After observing the increase in proliferation following mechanical strain, the isolated exosomes within each group were characterized to determine if mechanical strain impacted exosome production. By using a bicinchonicic acid (BCA) assay, no significant difference was observed in the protein concentration within the exosomes of each group following the mechanical strain regimens (FIG. 2D). However, the concentration of CD81+ exosomes were increased by both HSSD and LSLD regimens when compared to static conditions. While the HSSD conditions did not result in a significantly higher CD81+ exosome production (FIG. 2E, p=0.1468), LSLD conditions resulted in significantly increased exosomes relative to static (p<0.0001) and HSSD conditions (p=0.031, FIG. 2E). It is, thus, indicated that exercise causes not only cells to proliferate, but also causes an increased production of exosomes.

Using flow cytometry-based exosome quantification, the finding that exosome concentrations were increased was confirmed with a secondary measurement (FIG. 2F-2H). It is similarly shown herein that both HSSD and LSLD mechanical strain regimens resulted in the production of significantly more exosomes when compared to the static conditions (FIG. 2I, HSSD vs static p=0.0321, LSLD vs static p<0.0001, HSSD vs LSLD p=0.0351). Of note the two methods of exosome quantification were used to complement each other, with the difference in y-axis range when comparing FIGS. 2E and 2I likely due to the loss in single vesicle resolution through the flow cytometer.

Exosomes from Mechanically Strained C2C12 Cells Improve Proliferation and Differentiation

Following quantification, exosome suspensions from each group were added to naïve C2C12 cells. This resulted in a greater cell count for all groups treated with exosomes when compared to untreated cells (-Exo) (FIG. 48; F6,40=12.79, time and group comparisons found in FIG. 6). Following treatment with exosomes from the HSSD, a significant increase was observed in cell count compared to the untreated cells at days 3 and 5 (p=0.0004 and p=0.0004, respectively). Exosomes from the LSLD induced an even greater change, producing a significant difference between the LSLD and untreated cells at days 3 and 5 (p=0.0027 and p=0.0012, respectively). Exosomes isolated from the static group elicited no significant increase in cell count when compared to the untreated cells. When comparing the static group against HSSD and LSLD, no significant increase in cell count was found after treatment with HSSD exosomes; however, cells treated with LSLD exosomes were significantly more abundant at days 3 and 5 (p=0.0059 and p=0.0411, respectively). Lastly, cell count was significantly increased in time (p<0.0001) resulting in an improved growth rate for all cells treated with exosomes. The LSLD exosomes were found to be particularly effective imparting a 1.827-fold increase in growth rate while static and HSSD exosomes imparted a 0.366- and 1.018-fold increase, respectively (FIG. 4C).

Following proliferation, the exosome's effects on myogenic differentiation were investigated. All exosome suspensions resulted in greater myotube area and nuclear fusion indexes compared to untreated cells. The myotube area and nuclear fusion index for myotubes containing greater than 3 nuclei were significantly higher in exosomes from LSLD loading programs (FIGS. 4D-4I; p=0.0039 and p=0.0484, respectively).

Mechanical Strain Produces Unique and Differential miRNA within Exosomes

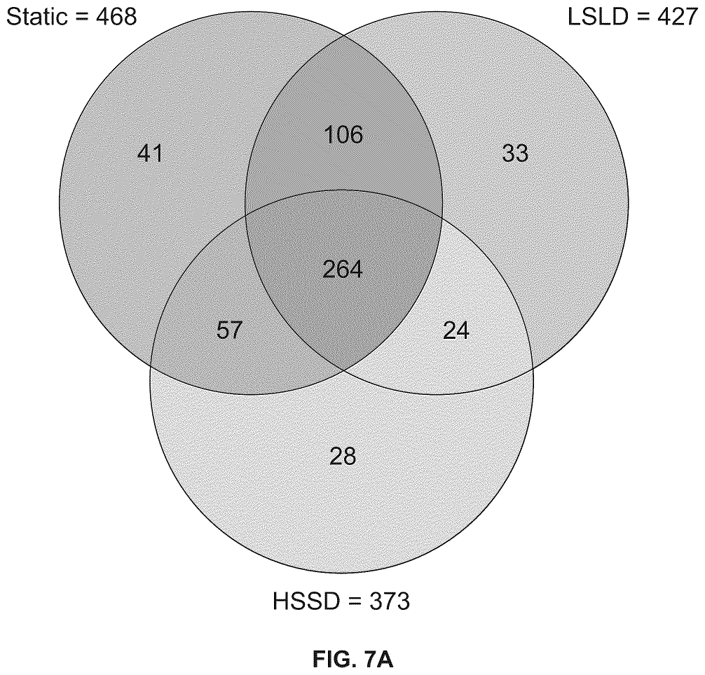

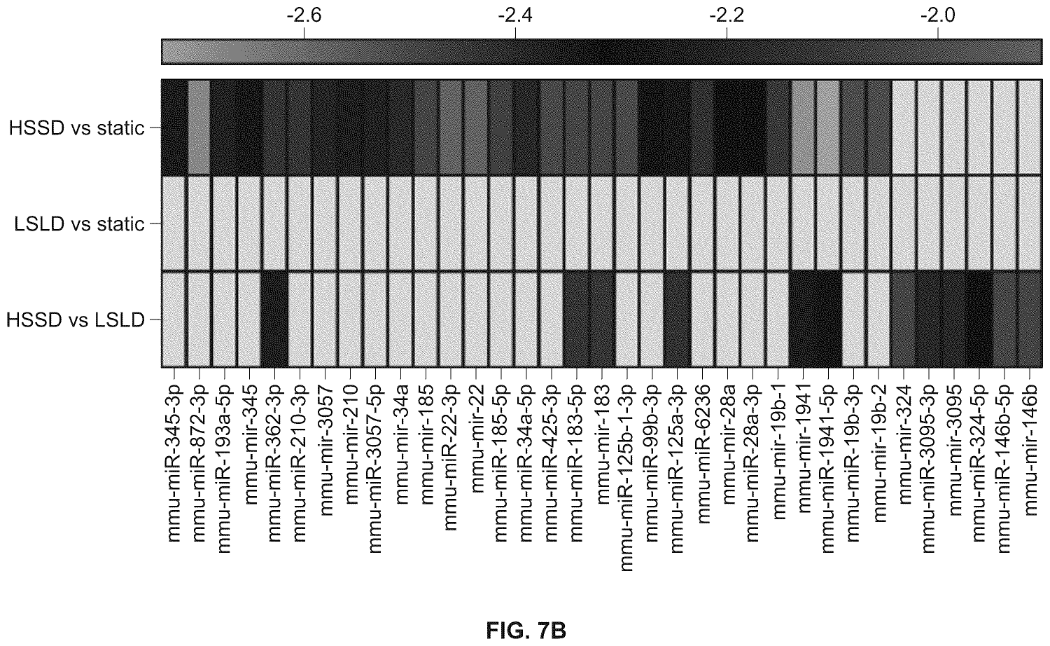

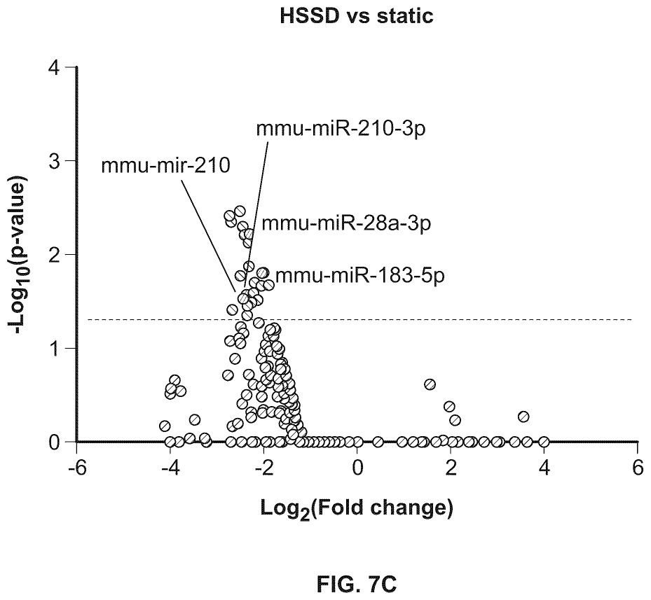

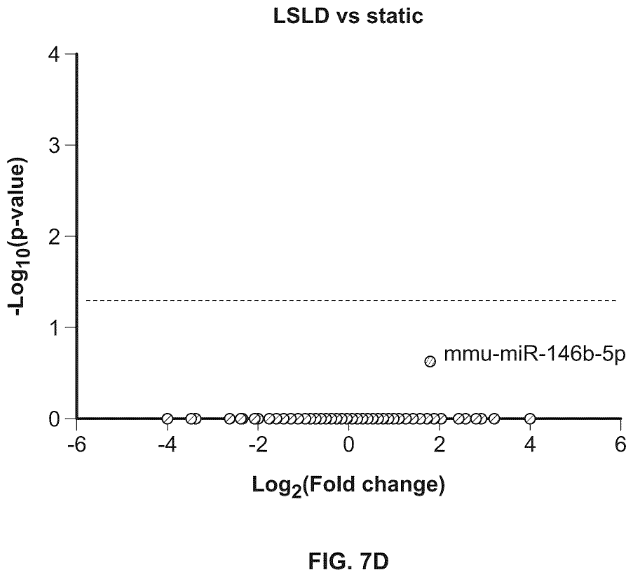

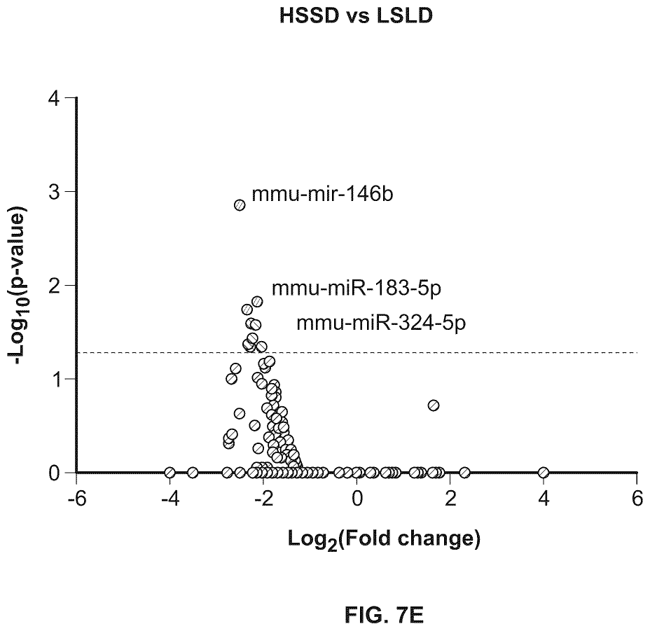

It was investigated whether differential expression of miRNA cargo within the exosomes from mechanically strained C2C12 cells might contribute to the observed functional changes in proliferation and myogenesis. miRNA sequencing results revealed that 553 precursor and mature miRNA strands were expressed across exosomes from all groups with both unique and overlapping miRNA content (FIG. 7A, Table 1, below). The static group contained the most overall identifiable miRNA (468), 41 more than LSLD (427) and 95 more than HSSD (373). 264 of the miRNAs were found across all groups, allowing focus upon a smaller subset of the uniquely expressed miRNAs. The static group had the highest number of unique miRNA (41), while LSLD and HSSD exosomes carried 33 and 28 unique miRNA, respectively. Static and LSLD exosomes had 106 shared miRNA, while static and HSSD shared 57 and LSLD and HSSD shared 24.

TABLE 1
All Mapped miRNA by Group

| HSSD | LSLD | SEQ ID NO |
|---|---|---|
| | mmu-mir-344b | 1 |
| | mmu-miR-3060-5p | 2 |
| | mmu-miR-344b-3p | 3 |
| mmu-mir-6378 | | 34 |
| mmu-miR-6378 | | 35 |
| | mmu-mir-12184 | 4 |
| | mmu-mir-293 | 5 |
| | mmu-miR-293-3p | 6 |
| | mmu-mir-804 | 7 |
| mmu-miR-6403 | | 36 |
| mmu-miR-7023-5p | | 37 |
| mmu-mir-6539 | | 38 |
| mmu-miR-1929-5p | | 39 |
| | mmu-miR-496a-3p | 8 |
| | mmu-miR-1930-5p | 9 |
| | mmu-mir-7119 | 10 |
| mmu-mir-3470b | | 40 |
| mmu-miR-3077-3p | | 41 |
| mmu-miR-491-3p | | 42 |
| mmu-mir-434 | | 43 |
| mmu-miR-128-2-5p | | 44 |
| mmu-miR-434-3p | | 45 |
| | mmu-miR-1950 | 11 |
| | mmu-mir-12201 | 12 |
| | mmu-miR-12201-5p | 13 |
| | mmu-miR-3109-3p | 14 |
| | mmu-miR-124-5p | |
| mmu-miR-496a-5p | | 46 |
| mmu-mir-12203 | | 47 |
| mmu-miR-12203-3p | | 48 |
| mmu-miR-363-5p | | 49 |
| | mmu-miR-7681-5p | 16 |
| | mmu-miR-103-1-5p | 17 |
| mmu-mir-1929 | | 50 |
| mmu-miR-7034-5p | | 51 |
| | mmu-miR-219c-3p | 18 |
| mmu-mir-6409 | | 52 |
| | mmu-miR-6961-5p | 19 |
| | mmu-miR-6244 | 20 |
| | mmu-miR-7065-5p | 21 |
| mmu-miR-5617-5p | | 53 |
| | mmu-miR-7675-5p | 22 |
| | mmu-miR-7117-3p | 23 |
| mmu-mir-7023 | | 54 |
| | mmu-miR-6546-3p | 24 |
| mmu-miR-2139 | | 55 |
| mmu-miR-3102-5p | | 56 |
| mmu-miR-871-3p | | 57 |
| mmu-miR-8108 | | 58 |
| mmu-miR-3073a-3p | | 59 |
| mmu-mir-5617 | | 60 |
| mmu-mir-871 | | 61 |
| | mmu-mir-7675 | 25 |
| | mmu-mir-7117 | 26 |
| | mmu-mir-6905 | 27 |
| | mmu-miR-6905-5p | 28 |
| | mmu-miR-5129-3p | 29 |
| | mmu-mir-7065 | 30 |
| | mmu-mir-1950 | 31 |
| | mmu-mir-5129 | 32 |
| | mmu-miR-365-1-5p | 33 |

35 miRNA had statistically significant (p<0.05) expression levels, with all 35 miRNAs being significantly downregulated in the HSSD exosomes when compared to exosomes from the static and LSLD groups (FIGS. 7B-7E). Compounding on these results, it was often found that when the precursor miRNA was found to be up- or downregulated, the mature miRNA was also found to be similarly expressed.

Differential Exosomal miRNA Regulate Specific Pathways

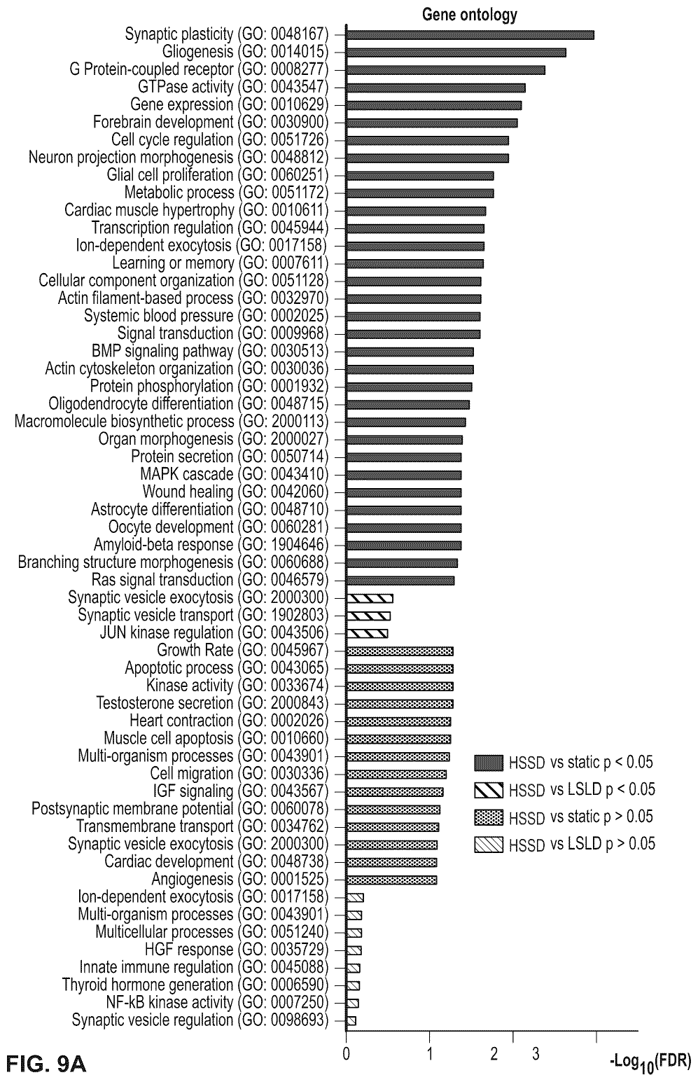

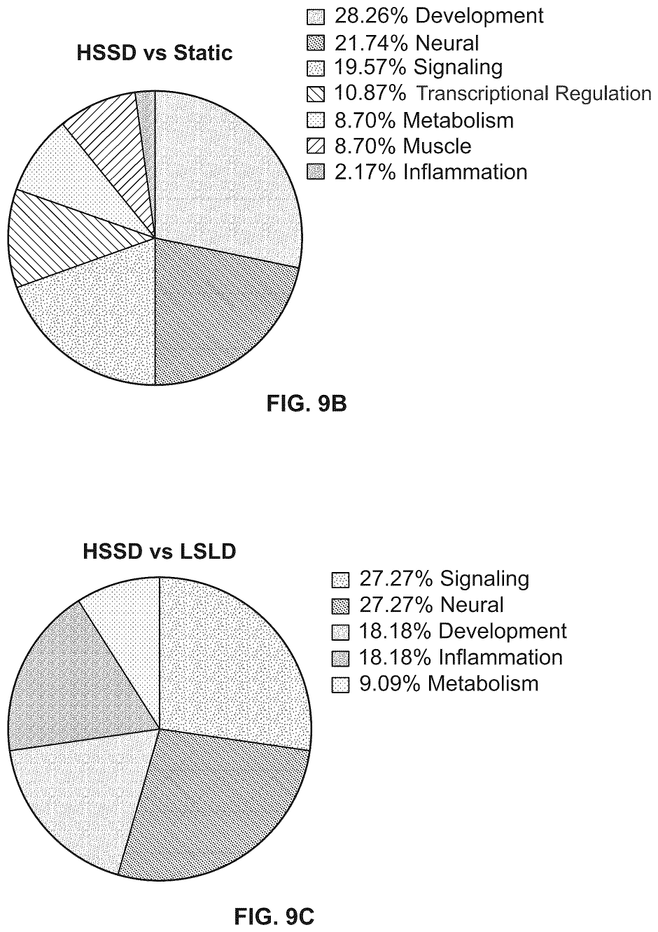

Upon gene ontology analysis, the significantly downregulated miRNAs in HSSD exosomes compared to static were associated with 218 potential gene targets, and these targets were enriched for 178 GO terms (Tables 2A (FIG. 8) and 2B, below). To find the most specific biological processes among these, terminal GO terms/nodes were identified. This process yielded 46 specific GO terms (FIG. 9A, solid bars) 32 of which were found to be significantly enriched (FDR<0.05, solid, blue bars). Regulation of BMP signaling, actin-filament based processes, and muscle cell apoptosis were noted and are programs highly associated with musculoskeletal development and repair.

TABLE 2b
HSSD v LSLD Enriched GO Terms
HSSD v LSLD Enriched GO Terms

| | | | FDR |
|---|---|---|---|
| GO Term | Description | P-value | q-value |
| GO: 0060627 | regulation of vesicle- | 2.68E−05 | 3.93E−01 |
| | mediated transport | | |
| GO: 0017157 | regulation of exocytosis | 4.92E−05 | 3.60E−01 |
| GO: 0099177 | regulation of trans- | 8.30E−05 | 4.06E−01 |
| | synaptic signaling | | |
| GO: 0050804 | modulation of chemical | 8.30E−05 | 3.04E−01 |
| | synaptic transmission | | |
| GO: 0051641 | cellular localization | 9.70E−05 | 2.85E−01 |
| GO: 0043506 | regulation of JUN kinase | 1.31E−04 | 3.19E−01 |
| | activity | | |
| GO: 1903305 | regulation of regulated | 1.35E−04 | 2.83E−01 |
| | secretory pathway | | |
| GO: 2000300 | regulation of synaptic | 1.51E−04 | 2.77E−01 |
| | vesicle exocytosis | | |
| GO: 1902803 | regulation of synaptic | 1.81E−04 | 2.96E−01 |
| | vesicle transport | | |
| GO: 0046928 | regulation of | 4.49E−04 | 6.59E−01 |
| | neurotransmitter secretion | | |
| GO: 0017158 | regulation of calcium | 4.65E−04 | 6.20E−01 |
| | ion-dependent exocytosis | | |
| GO: 0006590 | thyroid hormone generation | 5.73E−04 | 7.01E−01 |
| GO: 0098693 | regulation of synaptic | 6.67E−04 | 7.52E−01 |
| | vesicle cycle | | |
| GO: 0007250 | activation of NF-kappaB- | 6.86E−04 | 7.19E−01 |
| | inducing kinase activity | | |
| GO: 0035729 | cellular response to | 6.86E−04 | 6.71E−01 |
| | hepatocyte growth factor | | |
| | stimulus | | |
| GO: 0043900 | regulation of multi- | 7.53E−04 | 6.90E−01 |
| | organism process | | |
| GO: 0080134 | regulation of response | 7.79E−04 | 6.73E−01 |
| | to stress | | |
| GO: 0043901 | negative regulation of | 7.99E−04 | 6.51E−01 |
| | multi-organism process | | |
| GO: 0043549 | regulation of kinase activity | 8.78E−04 | 6.78E−01 |
| GO: 0045088 | regulation of innate | 9.31E−04 | 6.83E−01 |
| | immune response | | |
| GO: 0051240 | positive regulation of | 9.39E−04 | 6.56E−01 |
| | multicellular organismal | | |
| | process | | |
| GO: 0035728 | response to hepatocyte | 9.42E−04 | 6.28E−01 |
| | growth factor | | |

The downregulated miRNAs in HSSD exosomes compared to LSLD were associated with 53 gene targets (Tables 2A (FIG. 8) and 2B). In a GO analysis of these 53 gene targets, 22 GO terms were enriched (Tables 2A (FIG. 8) and 2B), and there were 11 specific processes/terminal GO terms yielded (FIG. 9A, striped bars), several of which suggested a role in synaptic vesicle processes. Of the 11 final GO terms, 3 were found to be significantly enriched (FDR<0.05, striped, blue bars).

Once categorized, the majority of enriched GO terms involved developmental processes (FIGS. 9B and 9C). Several other enriched GO terms suggested a general role in regulating processes associated with cellular signaling, metabolism, inflammation, and transcriptional regulation, suggesting that the exosomes found in this study had the ability to impart broad, systemic changes to a host of tissues and cell types (Tables 3A and 3B, below).

TABLE 3a
Gene Ontology Categories
HSSD vs Static

| Description | Reviewer 1 | Reviewer 2 | Reviewer 3 | Reviewer 4 | Final |
|---|---|---|---|---|---|
| Angiogenesis | Development | Development | Development | Development | Development |
| (GO: 0001525) | | | | | |
| Cardiac | Muscle | Muscle | Development | Muscle | Muscle |
| Development | | | | | |
| (GO: 0048738) | | | | | |
| Synaptic Vesicle | Signaling | Neural | Neural | Neural | Neural |
| Exocytosis | | | | | |
| (GO: 2000300) | | | | | |
| Transmembrane | Signaling | Signaling | Signaling | Signaling | Signaling |
| Transport | | | | | |
| (GO: 0034762) | | | | | |
| Postsynaptic | Signaling | Neural | Neural | Neural | Neural |
| Membrane | | | | | |
| Potential | | | | | |
| (GO: 0060078) | | | | | |
| IGF Signaling | Signaling | Signaling | Signaling | Signaling | Signaling |
| (GO: 0043567) | | | | | |
| Cell Migration | Development | Development | Development | Development | Development |
| (GO: 0030336) | | | | | |
| Multi-organism | Development | Development | Signaling | Development | Development |
| Processes | | | | | |
| (GO: 0043901) | | | | | |
| Muscle Cell | Muscle | Muscle | Muscle | Muscle | Muscle |
| Apoptosis | | | | | |
| (GO: 0010660) | | | | | |
| Heart | Muscle | Muscle | Muscle | Muscle | Muscle |
| Contraction | | | | | |
| (GO: 0002026) | | | | | |
| Testosterone | Signaling | Signaling | Signaling | Signaling | Signaling |
| Secretion | | | | | |
| (GO: 2000843) | | | | | |
| Kinase Activity | Metabolism | Metabolism | Signaling | Metabolism | Metabolism |
| (GO: 0033674) | | | | | |
| Apoptotic | Development | Development | Development | Development | Development |
| Process | | | | | |
| (GO: 0043065) | | | | | |
| Growth Rate | Development | Development | Development | Development | Development |
| (GO: 0045967) | | | | | |
| Ras Signal | Signaling | Signaling | Signaling | Signaling | Signaling |
| Transduction | | | | | |
| (GO: 0046579) | | | | | |
| Branching | Development | Development | Development | Development | Development |
| Structure | | | | | |
| Morphogenesis | | | | | |
| (GO: 0060688) | | | | | |
| Amyloid-Beta | Neural | Neural | Signaling | Neural | Neural |
| Response | | | | | |
| (GO: 1904646) | | | | | |
| Oocyte | Development | Development | Development | Development | Development |
| Development | | | | | |
| (GO: 0060281) | | | | | |
| Astrocyte | Neural | Neural | Neural | Neural | Neural |
| Differentiation | | | | | |
| (GO: 0048710) | | | | | |
| Wound Healing | Development | Inflammation | Inflammation | Inflammation | Inflammation |
| (GO: 0042060) | | | | | |
| MAPK Cascade | Signaling | Signaling | Signaling | Signaling | Signaling |
| (GO: 0043410) | | | | | |
| Protein Secretion | Signaling | Transcriptional | Transcriptional | Transcriptional | Transcriptional |
| (GO: 0050714) | | Regulation | Regulation | Regulation | Regulation |
| Organ | Development | Development | Development | Development | Development |
| Morphogenesis | | | | | |
| (GO: 2000027) | | | | | |
| Macromolecule | Development | Metabolism | Metabolism | Metabolism | Metabolism |
| Biosynthetic | | | | | |
| Process | | | | | |
| (GO: 2000113) | | | | | |
| Oligodendrocyte | Neural | Neural | Neural | Neural | Neural |
| Differentiation | | | | | |
| (GO: 0048715) | | | | | |
| Protein | Metabolism | Transcriptional | Transcriptional | Transcriptional | Transcriptional |
| Phosphorylation | | Regulation | Regulation | Regulation | Regulation |
| (GO: 0001932) | | | | | |
| Actin | Development | Development | Muscle | Development | Development |
| Cytoskeleton | | | | | |
| Organization | | | | | |
| (GO: 0030036) | | | | | |
| BMP Signaling | Development | Signaling | Development | Development | Development |
| Pathway | | | | | |
| (GO: 0030513) | | | | | |
| Signal | Signaling | Signaling | Signaling | Signaling | Signaling |
| Transduction | | | | | |
| (GO: 0009968) | | | | | |
| Systemic Blood | Signaling | Signaling | Inflammation | Signaling | Signaling |
| Pressure | | | | | |
| (GO: 0002025) | | | | | |
| Actin Filament- | Development | Development | Muscle | Development | Development |
| Based Process | | | | | |
| (GO: 0032970) | | | | | |
| Cellular | Development | Development | Development | Development | Development |
| Component | | | | | |
| Organization | | | | | |
| (GO: 0051128) | | | | | |
| Learning or | Development | Neural | Neural | Neural | Neural |
| Memory | | | | | |
| (GO: 0007611) | | | | | |
| Ion-dependent | Signaling | Signaling | Signaling | Signaling | Signaling |
| Exocytosis | | | | | |
| (GO: 0017158) | | | | | |
| Transcription | Metabolism | Transcriptional | Transcriptional | Transcriptional | Transcriptional |
| Regulation | | Regulation | Regulation | Regulation | Regulation |
| (GO: 0045944) | | | | | |
| Cardiac Muscle | Muscle | Muscle | Muscle | Muscle | Muscle |
| Hypertrophy | | | | | |
| (GO: 0010611) | | | | | |
| Metabolic | Metabolism | Metabolism | Metabolism | Metabolism | Metabolism |
| Process | | | | | |
| (GO: 0051172) | | | | | |
| Glial Cell | Neural | Neural | Neural | Neural | Neural |
| Proliferation | | | | | |
| (GO: 0060251) | | | | | |
| Neuron | Neural | Neural | Neural | Neural | Neural |
| Projection | | | | | |
| Morphogenesis | | | | | |
| (GO: 0048812) | | | | | |
| Cell Cycle | Metabolism | Transcriptional | Transcriptional | Transcriptional | Transcriptional |
| Regulation | | Regulation | Regulation | Regulation | Regulation |
| (GO: 0051726) | | | | | |
| Forebrain | Neural | Development | Development | Development | Development |
| Development | | | | | |
| (GO: 0030900) | | | | | |
| Gene Expression | Metabolism | Transcriptional | Transcriptional | Transcriptional | Transcriptional |
| (GO: 0010629) | | Regulation | Regulation | Regulation | Regulation |
| GTPase Activity | Metabolism | Metabolism | Signaling | Metabolism | Metabolism |
| (GO: 0043547) | | | | | |
| G Protein- | Signaling | Signaling | Signaling | Signaling | Signaling |
| coupled Receptor | | | | | |
| (GO: 0008277) | | | | | |
| Gliogenesis | Neural | Neural | Neural | Neural | Neural |
| (GO: 0014015) | | | | | |
| Synaptic | Neural | Neural | Neural | Neural | Neural |
| Plasticity | | | | | |
| (GO: 0048167) | | | | | |

TABLE 3b
HSSD v. LSLD
HSSD vs LSLD

| Description | Reviewer 1 | Reviewer 2 | Reviewer 3 | Reviewer 4 | Final |
|---|---|---|---|---|---|
| Synaptic Vesicle | Neural | Neural | Neural | Neural | Neural |
| Regulation | | | | | |
| (GO: 0098693) | | | | | |
| NF-kB Kinase | Inflammation | Inflammation | Inflammation | Inflammation | Inflammation |
| Activity | | | | | |
| (GO: 0007250) | | | | | |
| Thyroid Hormone | Metabolism | Metabolism | Metabolism | Metabolism | Metabolism |
| Generation | | | | | |
| (GO: 0006590) | | | | | |
| Innate Immune | Inflammation | Inflammation | Signaling | Inflammation | Inflammation |
| Regulation | | | | | |
| (GO: 0045088) | | | | | |
| HGF Response | Development | Signaling | Signaling | Signaling | Signaling |
| (GO: 0035729) | | | | | |
| Multicellular | Development | Development | Signaling | Development | Development |
| Processes | | | | | |
| (GO: 0051240) | | | | | |
| Multi-organism | Development | Development | Signaling | Development | Development |
| Processes | | | | | |
| (GO: 0043901) | | | | | |
| Ion-dependent: | Signaling | Signaling | Neural | Signaling | Signaling |
| Exocytosis | | | | | |
| (GO: 0017158 | | | | | |
| JUN Kinase | Signaling | Signaling | Signaling | Signaling | Signaling |
| Regulation | | | | | |
| (GO: 0043506) | | | | | |
| Synaptic Vesicle | Neural | Neural | Neural | Neural | Neural |
| Transport | | | | | |
| (GO: 1902803) | | | | | |
| Synaptic Vesicle | Neural | Neural | Neural | Neural | Neural |
| Exocytosis | | | | | |
| (GO: 2000300) | | | | | |

The health benefits of exercise are widely recognized, with exercise being an effective strategy to promote healthy aging by preventing several chronic diseases including metabolic disorders, cardiovascular disease, sarcopenia, and bone loss (Booth, et al., 2012 Compr Physiol 2 (2): 1143-1211; Garatachea, et al., 2015 Rejuvenation Res 18 (1): 57-89; Cartee, et al., 2016 Cell Metab 23 (6): 1034-1047; Beckwee, et al., 2019 J Nutr Heal Aging 23 (6): 494-452; Stefani and Galanti, 2017 Adv Exp Med Biol 123-141). Exercise improves overall health during aging, likely by a combination of molecular and biomechanical mechanisms, largely driven by preservation of skeletal muscle (Distefano and Goodpaster, 2018 Perspect Med 8 (3)). Complete understanding of this process has been impeded by complexities arising from the complex, whole-body-integrated effects of exercise, which obscure specific relationships between skeletal muscle, cartilage and bone health during aging (Kirby, et al., 2012 Circ Res 111 (2); Nicklas, et al., 2008 Aging Clin Exp Res 20 (4)). Dissecting such mechanisms could potentially lead to a targeted pharmaceutical therapy (as an adjunct to exercise) that could be especially efficacious in older individuals, where exercise is often challenging.

Although it was previously demonstrated that in vitro mechanical strain and in vivo treadmill exercise can further increase the regenerative capacity of muscle progenitor cells (Ambrosio, et al., 2010 Tissue Eng Pt A 16 (3): 839-849), the mechanism underlying this improvement is still unclear. While mechanical disruption of the cell membrane and influxes of calcium during skeletal muscle contraction have been shown to induce exosome release (Savina, et al., 2003 J Biol Chem 278 (22): 20083-20090; Bittel and Jaiswal, 2019 Front Physiol 10 https://pubmed.ncbi.nlm.nih.gov/31379590/), contradictions in the literature remain as to whether exercise stimulates an increase in skeletal muscle-derived exosomes released into the local tissue and into the bloodstream. In one study, there was no significant change in skeletal muscle exosomes in the bloodstream immediately following an hour of endurance exercise (Nielsen, et al., 2019 J Clin Endocrinol Metab 104 (10): 4804-4814). In contrast, a more recent study found an increase in skeletal muscle exosomes in circulation after 30 minutes of moderate exercise (Rigamonti, et al., 2020 Int J Obes 44 (5): 1108-1118), suggesting that skeletal muscle may partially contribute to the exercise-induced increase in circulating exosomes. Mechanical strain of myoblasts may cause the increased production of exosomes with pro-regenerative functions. The results described herein demonstrate that cyclical strain, an ex vivo exercise mimetic, results in increased production of C2C12 myoblast-derived exosomes with enhanced proliferative and myogenic functions.

Furthermore, the clinical application of mesenchymal stem cells (MSCs) remains limited due to regulatory hurdles and the lack of definitive guidelines for optimizing efficacy and safety. Good manufacturing practice (GMP)-grade stem cells are challenging to produce because of donor heterogeneity, passage limits and incompletely understood factors influencing their efficacy in different applications (Ährlund-Richter, et al., 2009 Cell Stem Cell 4 (1): 20-26; Truong, et al., 2019 Adv Exp Med Biol 1084:109-128), thus making it difficult to achieve uniformity between batches. These issues highlight the need for an alternative approach that can replicate the benefits of stem cell therapy, yet minimizing potential risks and regulatory challenges, with exosomes presenting a promising alternative.

Ultimately, the development of exosome-based therapeutics will require reproducible production of homogenous exosome suspension, which can be optimized for defined clinical applications. While in vitro cell culture systems do not fully recapitulate in vivo conditions, a scalable and highly reproducible in vitro exosome production platform could ultimately enable efficient development of GMP-grade exosome-based therapeutics. Exosomes hold promise in the area of regenerative medicine for several reasons (Cobelli, et al., 2017 Ann NY Acad Sci 1410 (1): 57-67; Tao, et al., 2018 Adv Sci 5 (2): 1700449). For instance, exosome surface receptors can be engineered for targeted delivery of cargo to specific tissues. In addition, exosomes can be modified to contain specific therapeutic cargo (i.e., regulatory RNAs, proteins, or drugs).

This study is innovative, in that an in vitro bioreactor was utilized to simulate the effects of mechanical strain on exosome production from myoblast cells during exercise. The results build upon the investigated changes in exosome production and miRNA expression from skeletal muscle in response to in vivo exercise (Gomes, et al., 2014 Biomarkers 19 (7): 585-589) by providing insight into the effects of mechanical stimulation on exosome production from myoblast progenitor cells. Upon gene ontology investigation, several differentially expressed miRNAs, including miR-146b-5p. miR-183-5p, miR-210-3p, and miR-324-5p, which have previously described roles in myogenesis (Lee, et al., 2020 BMC Mol Cell Biol 21 (1): 1-13; Liu, et al., 2020 Mol Ther Nucl Acids 22:722-732), osteogenesis (Xie, et al., 2017 Sci Reports 7 (1): 1-16), and cellular aging (Davis, et al., 2017 Tissue Eng—PL A 23 (21-22): 1231-1240) were identified in HSSD exosomes, suggesting that these miRNAs may also modulate musculoskeletal adaptations to mechanical strain. However, despite the HSSD exosomes showing differential expression of multiple miRNAs, this did not result in any functional changes being observed in the HSSD group in the current study. Exosomes transport diverse cargo comprised of not only miRNAs, but other DNA, RNAs, cytokines, proteins, and lipids (Kalluri and LeBleu, 2020 Science 80 (367): 6478), such additional exosomal cargo may contribute to the phenotypic changes observed in this study in addition to miRNAs. Furthermore, only 32.9% of all miRNAs found in this study were annotated with gene ontology terms.

While resistance exercise is currently the most effective strategy for the prevention of age-related muscle decline, collecting exercise-induced exosomes from serum is difficult, because circulatory exosomes represent a highly heterogeneous population from diverse tissue types (Vagner, et al., 2019 Proteomics 19 (8): 1800167). Thus, cell-type specific exosomes may be generated in vitro toward the goal of producing a clinically translatable exosome therapeutic to be used as a pre- (i.e. prehabilitation) or post- (i.e. rehabilitation) surgical intervention to aid in tissue repair and recovery. While physical therapy rehabilitation has long been known to improve musculoskeletal injuries and improve recovery timelines after surgery, prehabilitation is increasingly being utilized as a means to further improve the rehabilitation process (Jahic, et al., 2018 Med Arch 72 (6): 439-443; Carter, et al., 2020 BMC Musculoskeletal Disord 21 (1). Based on the instant findings, a major underlying component driving these effects of prehabilitation may in fact be mediated by the muscle-derived exosomes produced in response to exercise. These exosomes therefore hold the possibility of being administered to a patient as a prehabilitation treatment prior to surgery in place of exercise, to prepare the body for repair and recovery.

Given the many health benefits of exercise and the potential for exosomes to mediate these benefits, exercise-derived exosomes may have therapeutic applications. Indeed, exosomes that are released following exercise have recently been demonstrated to have cardioprotective (Bei, et al., 2017 Basic Res Cardiol 112 (4); Hou, et al., 2019 Circ Res 124 (9) and neuroprotective effects (Wang, et al., 2020 Exp Neurol 330). Mechanical strain is demonstrated herein to alter the number and function of myoblast-derived exosomes, and therefore may hold therapeutic promise for musculoskeletal repair and regeneration.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

1. A composition comprising exosomes comprising at least one mechanoresponsive miRNA.

2. The composition of claim 1, wherein the exosomes are derived from mechanosensitive cells.

3. (canceled)

4. (canceled)

5. The composition of claim 1, wherein the exosomes are isolated from a cell sample after ex vivo exercise.

6. The composition of claim 5, wherein the exercise is achieved by a bioreactor creating mechanical strain on cells in the sample.

7. The composition of claim 5, wherein the exercise is low intensity or high intensity.

8. The composition of claim 1, wherein the exosomes are artificially synthesized.

9. The composition of claim 1, wherein the at least one mechanoresponsive miRNA is pro-regenerative.

10. The composition of claim 1, wherein the at least one mechanoresponsive miRNA is the human equivalent of an miRNA selected from the group consisting of: mmu-mir-344b (SEQ ID NO: 1), mmu-miR-3060-5p (SEQ ID NO: 2), mmu-miR-344b-3p (SEQ ID NO: 3), mmu-mir-12184 (SEQ ID NO: 4), mmu-mir-293 (SEQ ID NO: 5), mmu-miR-293-3p (SEQ ID NO: 6), mmu-mir-804 (SEQ ID NO: 7), mmu-miR-496a-3p (SEQ ID NO: 8), mmu-miR-1930-5p (SEQ ID NO: 9), mmu-mir-7119 (SEQ ID NO: 10), mmu-miR-1950 (SEQ ID NO: 11), mmu-mir-12201 (SEQ ID NO: 12), mmu-miR-12201-5p (SEQ ID NO: 13), mmu-miR-3109-3p (SEQ ID NO: 14), mmu-miR-124-5p (SEQ ID NO: 15), mmu-miR-7681-5p (SEQ ID NO: 16), mmu-miR-103-1-5p (SEQ ID NO: 17), mmu-miR-219c-3p (SEQ ID NO: 18), mmu-miR-6961-5p (SEQ ID NO: 19), mmu-miR-6244 (SEQ ID NO: 20), mmu-miR-7065-5p (SEQ ID NO: 21), mmu-miR-7675-5p (SEQ ID NO: 22), mmu-miR-7117-3p (SEQ ID NO: 23, mmu-miR-6546-3p (SEQ ID NO: 24), mmu-mir-7675 (SEQ ID NO: 25), mmu-mir-7117 (SEQ ID NO: 26), mmu-mir-6905 (SEQ ID NO: 27), mmu-miR-6905-5p (SEQ ID NO: 28), mmu-miR-5129-3p (SEQ ID NO: 29), mmu-mir-7065 (SEQ ID NO: 30), mmu-mir-1950 (SEQ ID NO: 31), mmu-mir-5129 (SEQ ID NO: 32), mmu-miR-365-1-5p (SEQ ID NO: 33), mmu-mir-6378 (SEQ ID NO: 34), mmu-miR-6378 (SEQ ID NO: 35), mmu-miR-6403 (SEQ ID NO: 36), mmu-miR-7023-5p (SEQ ID NO: 37), mmu-mir-6539 (SEQ ID NO: 38), mmu-miR-1929-5p (SEQ ID NO: 39), mmu-mir-3470b (SEQ ID NO: 40, mmu-miR-3077-3p (SEQ ID NO: 41), mmu-miR-491-3p (SEQ ID NO: 42), mmu-mir-434 (SEQ ID NO: 43), mmu-miR-128-2-5p (SEQ ID NO: 44), mmu-miR-434-3p (SEQ ID NO: 45), mmu-miR-496a-5p (SEQ ID NO: 46), mmu-mir-12203 (SEQ ID NO: 47, mmu-miR-12203-3p (SEQ ID NO: 48), mmu-miR-363-5p (SEQ ID NO: 49), mmu-mir-1929 (SEQ ID NO: 50), mmu-miR-7034-5p (SEQ ID NO: 51), mmu-mir-6409 (SEQ ID NO: 52), mmu-miR-5617-5p (SEQ ID NO: 53), mmu-mir-7023 (SEQ ID NO: 54), mmu-miR-2139 (SEQ ID NO: 55), mmu-miR-3102-5p (SEQ ID NO: 56), mmu-miR-871-3p (SEQ ID NO: 57), mmu-miR-8108 (SEQ ID NO: 58), mmu-miR-3073a-3p (SEQ ID NO: 59), mmu-mir-5617 (SEQ ID NO: 60), and mmu-mir-871 (SEQ ID NO: 61).

11. The composition of claim 10, wherein the at least one mechanoresponsive miRNA is the human equivalent of an miRNA selected from the group consisting of: mmu-mir-344b (SEQ ID NO: 1), mmu-miR-3060-5p (SEQ ID NO: 2), mmu-miR-344b-3p (SEQ ID NO: 3), mmu-mir-12184 (SEQ ID NO: 4), mmu-mir-293 (SEQ ID NO: 5), mmu-miR-293-3p (SEQ ID NO: 6), mmu-mir-804 (SEQ ID NO: 7), mmu-miR-496a-3p (SEQ ID NO: 8), mmu-miR-1930-5p (SEQ ID NO: 9), mmu-mir-7119 (SEQ ID NO: 10), mmu-miR-1950 (SEQ ID NO: 11), mmu-mir-12201 (SEQ ID NO: 12), mmu-miR-12201-5p (SEQ ID NO: 13), mmu-miR-3109-3p (SEQ ID NO: 14), mmu-miR-124-5p (SEQ ID NO: 15), mmu-miR-7681-5p (SEQ ID NO: 16), mmu-miR-103-1-5p (SEQ ID NO: 17), mmu-miR-219c-3p (SEQ ID NO: 18), mmu-miR-6961-5p (SEQ ID NO: 19), mmu-miR-6244 (SEQ ID NO: 20), mmu-miR-7065-5p (SEQ ID NO: 21), mmu-miR-7675-5p (SEQ ID NO: 22), mmu-miR-7117-3p (SEQ ID NO: 23), mmu-miR-6546-3p (SEQ ID NO: 24, mmu-mir-7675 (SEQ ID NO: 25), mmu-mir-7117 (SEQ ID NO: 26), mmu-mir-6905 (SEQ ID NO: 27), mmu-miR-6905-5p (SEQ ID NO: 28), mmu-miR-5129-3p (SEQ ID NO: 29), mmu-mir-7065 (SEQ ID NO: 30), mmu-mir-1950 (SEQ ID NO: 31), mmu-mir-5129 (SEQ ID NO: 32), and mmu-miR-365-1-5p (SEQ ID NO: 33).

12. The composition of claim 10, wherein the at least one mechanoresponsive miRNA is the human equivalent of an miRNA selected from the group consisting of: mmu-mir-6378 (SEQ ID NO: 34), mmu-miR-6378 (SEQ ID NO: 35), mmu-miR-6403 (SEQ ID NO: 36), mmu-miR-7023-5p (SEQ ID NO: 37), mmu-mir-6539 (SEQ ID NO: 38, mmu-miR-1929-5p (SEQ ID NO: 39), mmu-mir-3470b (SEQ ID NO: 40), mmu-miR-3077-3p (SEQ ID NO: 41), mmu-miR-491-3p (SEQ ID NO: 42), mmu-mir-434 (SEQ ID NO: 43), mmu-miR-128-2-5p (SEQ ID NO: 44), mmu-miR-434-3p (SEQ ID NO: 45), mmu-miR-496a-5p (SEQ ID NO: 46), mmu-mir-12203 (SEQ ID NO: 47), mmu-miR-12203-3p (SEQ ID NO: 48), mmu-miR-363-5p (SEQ ID NO: 49), mmu-mir-1929 (SEQ ID NO: 50), mmu-miR-7034-5p (SEQ ID NO: 51), mmu-mir-6409 (SEQ ID NO: 52), mmu-miR-5617-5p (SEQ ID NO: 53), mmu-mir-7023 (SEQ ID NO: 54), mmu-miR-2139 (SEQ ID NO: 55), mmu-miR-3102-5p (SEQ ID NO: 56), mmu-miR-871-3p (SEQ ID NO: 57), mmu-miR-8108 (SEQ ID NO: 58), mmu-miR-3073a-3p (SEQ ID NO: 59, mmu-mir-5617 (SEQ ID NO: 60), and mmu-mir-871 (SEQ ID NO: 61).

13. The composition of claim 1 for use in promoting regeneration in a subject.

14. The composition of claim 1 for use in the treatment of a musculoskeletal condition or disorder.

15. A method of preparing a composition comprising exosomes comprising at least one mechanoresponsive miRNA, the method comprising subjecting a mammal to exercise, obtaining a biological sample from the mammal, and isolating the exosomes from the sample.

16. A method of preparing a composition comprising exosomes comprising at least one mechanoresponsive miRNA, the method comprising exercising cells ex vivo and isolating the exosomes from the cells.

17. The method of claim 16, wherein the exercising is achieved by a bioreactor creating mechanical strain on the cells.

18. The method of claim 15, wherein the exercising is at low intensity or high intensity.

19. The method of claim 15, wherein the at least one mechanoresponsive miRNA is pro-regenerative.

20. The method of claim 15, wherein the at least one mechanoresponsive miRNA is the human equivalent of an miRNA selected from the group consisting of: mmu-mir-344b (SEQ ID NO: 1), mmu-miR-3060-5p (SEQ ID NO: 2, mmu-miR-344b-3p (SEQ ID NO: 3), mmu-mir-12184 (SEQ ID NO: 4), mmu-mir-293 (SEQ ID NO: 5), mmu-miR-293-3p (SEQ ID NO: 6), mmu-mir-804 (SEQ ID NO: 7), mmu-miR-496a-3p (SEQ ID NO: 8), mmu-miR-1930-5p (SEQ ID NO: 9), mmu-mir-7119 (SEQ ID NO: 10), mmu-miR-1950 (SEQ ID NO: 11), mmu-mir-12201 (SEQ ID NO: 12), mmu-miR-12201-5p (SEQ ID NO: 13), mmu-miR-3109-3p (SEQ ID NO: 14), mmu-miR-124-5p (SEQ ID NO: 15), mmu-miR-7681-5p (SEQ ID NO: 16), mmu-miR-103-1-5p (SEQ ID NO: 17), mmu-miR-219c-3p (SEQ ID NO: 18), mmu-miR-6961-5p (SEQ ID NO: 19), mmu-miR-6244 (SEQ ID NO: 20), mmu-miR-7065-5p (SEQ ID NO: 21, mmu-miR-7675-5p (SEQ ID NO: 22), mmu-miR-7117-3p (SEQ ID NO: 23), mmu-miR-6546-3p (SEQ ID NO: 24), mmu-mir-7675 (SEQ ID NO: 25), mmu-mir-7117 (SEQ ID NO: 26), mmu-mir-6905 (SEQ ID NO: 27), mmu-miR-6905-5p (SEQ ID NO: 28), mmu-miR-5129-3p (SEQ ID NO: 29), mmu-mir-7065 (SEQ ID NO: 30), mmu-mir-1950 (SEQ ID NO: 31), mmu-mir-5129 (SEQ ID NO: 32), mmu-miR-365-1-5p (SEQ ID NO: 33, mmu-mir-6378 (SEQ ID NO: 34), mmu-miR-6378 (SEQ ID NO: 35), mmu-miR-6403 (SEQ ID NO: 36), mmu-miR-7023-5p (SEQ ID NO: 37), mmu-mir-6539 (SEQ ID NO: 38), mmu-miR-1929-5p (SEQ ID NO: 39), mmu-mir-3470b (SEQ ID NO: 40), mmu-miR-3077-3p (SEQ ID NO: 41), mmu-miR-491-3p (SEQ ID NO: 42), mmu-mir-434 (SEQ ID NO: 43), mmu-miR-128-2-5p (SEQ ID NO: 44), mmu-miR-434-3p (SEQ ID NO: 45), mmu-miR-496a-5p (SEQ ID NO: 46), mmu-mir-12203 (SEQ ID NO: 47), mmu-miR-12203-3p (SEQ ID NO: 48), mmu-miR-363-5p (SEQ ID NO: 49), mmu-mir-1929 (SEQ ID NO: 50), mmu-miR-7034-5p (SEQ ID NO: 51), mmu-mir-6409 (SEQ ID NO: 52), mmu-miR-5617-5p (SEQ ID NO: 53), mmu-mir-7023 (SEQ ID NO: 54), mmu-miR-2139 (SEQ ID NO: 55), mmu-miR-3102-5p (SEQ ID NO: 56), mmu-miR-871-3p (SEQ ID NO: 57), mmu-miR-8108 (SEQ ID NO: 58), mmu-miR-3073a-3p (SEQ ID NO: 59, mmu-mir-5617 (SEQ ID NO: 60), and mmu-mir-871 (SEQ ID NO: 61).

21. The method of claim 20, wherein the at least one mechanoresponsive miRNA is the human equivalent of an miRNA selected from the group consisting of: mmu-mir-344b (SEQ ID NO: 1), mmu-miR-3060-5p (SEQ ID NO: 2), mmu-miR-344b-3p (SEQ ID NO: 3), mmu-mir-12184 (SEQ ID NO: 4), mmu-mir-293 (SEQ ID NO: 5), mmu-miR-293-3p (SEQ ID NO: 6), mmu-mir-804 (SEQ ID NO: 7), mmu-miR-496a-3p (SEQ ID NO: 8), mmu-miR-1930-5p (SEQ ID NO: 9), mmu-mir-7119 (SEQ ID NO: 10), mmu-miR-1950 (SEQ ID NO: 11), mmu-mir-12201 (SEQ ID NO: 12), mmu-miR-12201-5p (SEQ ID NO: 13), mmu-miR-3109-3p (SEQ ID NO: 14), mmu-miR-124-5p (SEQ ID NO: 15), mmu-miR-7681-5p (SEQ ID NO: 16), mmu-miR-103-1-5p (SEQ ID NO: 17), mmu-miR-219c-3p (SEQ ID NO: 18), mmu-miR-6961-5p (SEQ ID NO: 19), mmu-miR-6244 (SEQ ID NO: 20), mmu-miR-7065-5p (SEQ ID NO: 21), mmu-miR-7675-5p (SEQ ID NO: 22), mmu-miR-7117-3p (SEQ ID NO: 23), mmu-miR-6546-3p (SEQ ID NO: 24, mmu-mir-7675 (SEQ ID NO: 25), mmu-mir-7117 (SEQ ID NO: 26), mmu-mir-6905 (SEQ ID NO: 27), mmu-miR-6905-5p (SEQ ID NO: 28), mmu-miR-5129-3p (SEQ ID NO: 29), mmu-mir-7065 (SEQ ID NO: 30), mmu-mir-1950 (SEQ ID NO: 31), mmu-mir-5129 (SEQ ID NO: 32), and mmu-miR-365-1-5p (SEQ ID NO: 33).

22. The method of claim 20, wherein the at least one mechanoresponsive miRNA is the human equivalent of an miRNA selected from the group consisting of: mmu-mir-6378 (SEQ ID NO: 34), mmu-miR-6378 (SEQ ID NO: 35), mmu-miR-6403 (SEQ ID NO: 36), mmu-miR-7023-5p (SEQ ID NO: 37), mmu-mir-6539 (SEQ ID NO: 38), mmu-miR-1929-5p (SEQ ID NO: 39), mmu-mir-3470b (SEQ ID NO: 40), mmu-miR-3077-3p (SEQ ID NO: 41), mmu-miR-491-3p (SEQ ID NO: 42), mmu-mir-434 (SEQ ID NO: 43), mmu-miR-128-2-5p (SEQ ID NO: 44), mmu-miR-434-3p (SEQ ID NO: 45), mmu-miR-496a-5p (SEQ ID NO: 46), mmu-mir-12203 (SEQ ID NO: 47), mmu-miR-12203-3p (SEQ ID NO: 48), mmu-miR-363-5p (SEQ ID NO: 49), mmu-mir-1929 (SEQ ID NO: 50, mmu-miR-7034-5p (SEQ ID NO: 51), mmu-mir-6409 (SEQ ID NO: 52), mmu-miR-5617-5p (SEQ ID NO: 53), mmu-mir-7023 (SEQ ID NO: 54), mmu-miR-2139 (SEQ ID NO: 55), mmu-miR-3102-5p (SEQ ID NO: 56), mmu-miR-871-3p (SEQ ID NO: 57), mmu-miR-8108 (SEQ ID NO: 58), mmu-miR-3073a-3p (SEQ ID NO: 59), mmu-mir-5617 (SEQ ID NO: 60), and mmu-mir-871 (SEQ ID NO: 61).

23. (canceled)

24. (canceled)
